(19) United States
(12) Patent Application Publication
(10) Pub. No.: US 2023/0413807 A1
(43) Pub. Date: Dec. 28, 2023

(54) METHODS OF STABLE VITRIFICATION

(71) Applicant: UPKARA, INC., Ann Arbor, MI (US)

(72) Inventors: Pravansu Mohanty, Ann Arbor, MI (US); Mary Retzlaff, Ann Arbor, MI (US); Laura Bronsat, Ann Arbor, MI (US); Yolanda Taverner, Ann Arbor, MI (US); Zhouran Wang, Ann Arbor, MI (US); Animesh Koneru, Ann Arbor, MI (US)

(21) Appl. No.: 18/253,076

(22) PCT Filed: Nov. 19, 2021
(86) PCT No.: PCT/US2021/060164

§ 371 Date: May 16, 2023

Related U.S. Application Data
(60) Provisional application No. 63/115,936, filed on Nov. 19, 2020.

(51) Int. Cl. A01N 1/02

(52) U.S. Cl. CPC A01N 1/0284; A01N 1/0289

(57) ABSTRACT

Disclosed are methods for non-cryogenic vitrification of biological materials that include the steps of providing a biological sample and vitrification medium on a capillary substrate network within a desiccation chamber and providing both a heat energy and a lowered atmospheric pressure to provide for rapid vitrification without the vitrification medium or biological sample exhibiting cryogenic temperature or boiling as a result of lowered atmospheric pressure.

### RELATED APPLICATIONS

This application claims priority to U.S. Provisional Patent Application 63/115,936, file Nov. 19, 2020, the contents of which are hereby incorporated by reference in its entirety.

### FIELD

The present disclosure concerns methods for vitrification and preparation of biological materials to be capable of stable ambient temperature storage.

### BACKGROUND

Vitrification is the process of direct transition from a liquid to an amorphous glassy state. The process of vitrification is often applied to preserve biological materials and historically involves cooling the biological materials to cryogenic temperatures either intentionally or due to exposure to reduced atmospheric pressures. At cryogenic temperatures, vitrification techniques may lead to exposure to some of the damaging effects that normally occur with cryogenic storage due to ice crystal formation, which are known to form during conventional cryopreservation. To help address this issue, cryogenic protectants (CPAs) are used at high concentrations to avoid ice nucleation. Lower concentrations of CPAs are desired but in order to achieve preservation at these lower concentrations, ultra-fast heat transfer is required.

Heat transfer rates can be increased by reducing the sample volume and/or by increasing the cooling rate. A number of techniques have been utilized to increase the cooling rate such as employing thin straws or ultra-thin films to minimize the volume to be vitrified.

Anhydrous vitrification at ambient temperatures is an alternative strategy for preserving biological materials. In nature, a wide variety of organisms can survive extreme dehydration, which correlates in many cases with the accumulation of large amounts (as much as 20% of their dry weight) of glass forming sugars such as trehalose and sucrose in intracellular space. Such “glass forming” sugars historically need to be present on both sides of the plasma membrane to provide protection against the damaging effects of desiccation. Desiccation techniques dramatically limit or arrest the material's biochemical processes in a glassy matrix. Despite the success in vitrifying many biological materials such as proteins by anhydrous vitrification, broader applications to cellular materials still require one to increase the desiccation tolerance of the cells themselves.

Methods to enhance desiccation tolerance include utilizing improved vitrification medium containing trehalose, glycerol, pullulan and sucrose. While improved methods for loading cells with protective agents are helpful, there is a further need to develop techniques to minimize cellular injury during desiccation. Injury and degradation may result from the high sensitivity of cells in general to prolonged exposure to osmotic stress during dry processing. Osmotic stress can cause cell death at relatively high moisture content even in the presence of protective sugars like trehalose.

The most common approach to desiccating cells involves drying in sessile droplets with suspended cells. However, desiccation using evaporative drying of sessile droplets is inherently slow and non-uniform in nature. A glassy skin forms at the liquid/vapor interface of the sample when the cells are desiccated in glass forming solutions. This glassy skin slows and ultimately prevents further desiccation of the sample beyond a certain level of dryness and induces significant spatial non-uniformity of the water content across the sample. As a result, cells trapped in the partially desiccated sample underneath the glassy skin may not vitrify but degrade due to high molecular mobility.

The development of a fast and practical desiccation technique to achieve very low and uniform final moisture levels across the sample might overcome the shortcomings of the anhydrous vitrification techniques. Dry preservation suffers from a major limitation in long-term storage due to the degradation of the biological material by cumulative chemical stresses encountered as the vitrification solution gets concentrated in the extra-cellular space.

Prior techniques for desiccation have applied a vacuum to rapidly dry the cells at a temperature below the glass transition temperature. However, the application of the vacuum can significantly lower the temperature of the biological sample such that the sample experiences ice crystal formation, which can lead to cell damage. Furthermore, the atmospheric change provided by a vacuum also affects the boiling point of materials therein, providing a further risk to damaging the materials. This disclosure provides fast desiccation methods that produce improved viability of vitrified biological material through avoidance of freezing within the sample and without solution boiling to as to significantly facilitate long term storage of biological materials at non-cryogenic temperatures as well as overcome the challenges associated with prior cryogenic vitrification and storage technologies.

### BRIEF DESCRIPTION OF THE DRAWINGS

The drawings are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present disclosure. Exemplary aspects will become more fully understood from the detailed description and the accompanying drawings, wherein:

FIG. 1A shows a hydrophilic bed 10 with a thin film of liquid 20 placed a top where the capillary force is significantly higher than the viscous force. This limits the amount of liquid that can be desiccated 21.

FIG. 1B shows a substrate that is shaped into a contoured capillary bed, wherein desiccation can preferentially occur at the peaks of the contours 30 where capillary phenomena from the troughs toward the peaks during desiccation can enhance overall vitrification rate and allow for vitrification of large sample volumes relative to FIG. 1A.

FIG. 1C shows liquid filling the surface patterns when there is excess fluid within the contoured capillaries 40, resulting in bubble nucleation and boiling becoming dominant under reduced pressure which may lead to damage of sensitive molecules.

FIG. 2A shows a generalized schematic of vitrification. Cryogenic vitrification is historically achieved by fast cooling a liquid (pathway 1-2-3) (containing biological or other materials) to below the glass transition temperature bypassing the freezing zone. The total mass of the material is conserved through the process. Similarly, vitrification of materials can be achieved by fast desiccation bypassing the crystallization process (pathway 1-5-6). In this case, significant mass loss (primarily water) occurs. Cryogenic vitrification of a large amount material can be challenging due to heat transfer limitations and hence generally carried out in vials that provide significant surface/volume ratio. Similarly, fast desiccation is facilitated by large surface/volume ratio and specifically at reduced pressure. Reduction of pressure also reduces the boiling point of the liquid, which risks undesirable boiling of the liquid while vitrifying sensitive biomolecules or materials. The pathway of 1-4-6 shows the schematic of the present disclosure, where the application of heat and low atmospheric pressure allow for fast vitrification avoiding experience of freezing temperatures.

FIG. 2B shows a schematic of the triple point for water with a targeted sample temperature T₁ avoiding the triple point and hence freezing during vitrification.

FIG. 3 shows an exemplary capillary substrate (membrane) to facilitate fast desiccation of larger volume of liquid under vacuum to form glass.

FIG. 4 at (A) shows that when excess liquid accumulates on the surface of the capillary substrate, the capillary effect is not realized and under vacuum, boiling still can occur in the accumulated liquid, which is undesirable. To realize the capillary effect the liquid may be accommodated within the pores of the capillary substrate forming a meniscus. This localization on an undulating surface is similar and shaped by the peaks and troughs of the material. The liquid fraction (ξ) at the capillary interface, i.e., the area (in 2 dimensional schematic) occupied by liquid is one parameter that allows for optimization of capillary evaporation. Capillary driven evaporation occurs when the viscous pressure drop in the liquid surpasses the maximum capillary pressure at the liquid-vapor interface. The liquid fraction ξ is related to the overall pressure drop from the bulk to the liquid-vapor interface. Under atmospheric pressure and no applied heat flux (B) the liquid covers large area, leading to a liquid fraction, ξ→1. Under these conditions the capillary driven evaporation rate is minimal. Reducing the ambient pressure as shown in (C), reduces ξ and in turn increases the evaporation rate. However, beyond certain threshold pressure drop, nucleation boiling can occur which is undesirable. An applied heat flux Q as shown in (D) can also enhance the evaporation rate, but the risk of film boiling exists, which is also undesirable. Applying the heat flux from the surface of the capillary meniscus as shown in (E), eliminates the risk of film boiling. Large ΔP and Q applied in a counter gradient fashion as shown in (F), leads to the liquid meniscus confined to the pores, i.e., the liquid fraction ξ<<1 (e.g. ˜0.25), resulting in highest evaporation rate while avoiding boiling. Therefore, maintaining a temperature gradient between the surface and the bulk liquid leads to capillary evaporation as illustrated in (F), where the fast evaporation can be achieved. As the liquid level recedes into the capillary substrate, capillary evaporation phenomena is still realized as long as the pressure gradient and temperature gradients are maintained.

FIG. 5 at (A) shows exemplary results for glass membranes (capillary substrates) of different shapes loaded with a liquid containing 4% BSA, 15% Trehalose Dihydrate, 0.75% Glycerol, 2% Tween-20, and water. The liquid loading per mm² of the membrane was kept at 0.316 ml for all cases. For Case 1, the membranes were cut into 0.25 inch diameter circles and each was loaded with 10 ml liquid. A total of 48 samples containing 480 ml was loaded on a heated (37° C.) wire mesh inside a vacuum chamber. For Case 2, three long membrane strips (240 mmx 6.23 mm) each containing 470 ml liquid were loaded on the heated wire mesh. For case 3, a single strip (240 mm×22 mm) containing 1700 ml liquid was utilized. The chamber was evacuated to 29.5 mmHg. The temperature-time plots indicate the stages of the vitrification process. At the onset of the evacuation process, the pressure drops quickly while the membranes scaffold contains mostly liquid and as expected the temperature drops with the pressure drops. The supplied heat flux from the wire mesh/bed prevents further drop of the scaffold temperature into freezing regime. It is to noted that the freezing point can extend into subzero temperatures depending on the formulation of the vitrification excipient/liquid. Besides preventing freezing, the supplied heat flux from the bed also facilitates capillary evaporation while preventing boiling of the liquid under reduced pressure as illustrated above (FIG. 4 at F). As the moisture evaporates from the scaffold, the temperature rises until it reaches the bed temperature. The heat flux is controlled so that the scaffold temperature doesn't go above a set temperature, usually the bed temperature. As seen from FIG. 5, the time taken for the membrane scaffold temperature to reach the bed temperature varies with the scaffold configuration as well as the amount of liquid loaded onto it. The time taken to reach the bed temperature is a measure of the primary vitrification time meaning majority of the liquid is evaporated in this period. However, desiccation process still may prolong beyond this period to remove some residual moisture, which can be termed as secondary desiccation, which is not dependent the capillary phenomena. The process parameters and the scaffold geometry are chosen to optimize the volume of the liquid that can undergo primary desiccation process in a given time. In general, faster desiccation rate is desirable to bypass the crystal precipitation phase boundary indicated in FIG. 2A and to ensure glass formation. However, a threshold rate above which vitrification is ensured depends on the chemistry of the liquid, the capillary substrate characteristics such as hydrophilicity, porosity and dimensions. Temperature profile for a vitrification cycle (B) performed using the capillary network vitrification scaffold (solid trace) and a non-porous glass surface (dotted lines) and residual moisture levels (open circles) obtained for the scaffold sample. The temperature fluctuations seen using the glass support in the 1-6 minute time window were recorded concurrently with the observation that the liquid was bubbling and appeared to boil. No bubbling was observed for the sample vitrified on the scaffold.

FIG. 6A illustrates one exemplary aspect of the present disclosure wherein the desiccation device itself features contoured walls. The desiccation device can be formed of a hydrophilic capillary substrate (membrane) rolled into a cylindrical shape. The cylinder can house a vitrification medium within the membrane similar to FIG. 4 thereby promoting improved vitrification.

FIG. 6B shows a further aspect of the present disclosure, wherein a porous material capillary substrate is placed within a cylinder that can operably connect to a vacuum and sealed for vitrification of a sample placed on the membrane, with the membrane providing the contiguous capillary network for vitrification.

FIG. 7A illustrates one exemplary aspect of the present disclosure wherein the cylindrical desiccation devices are placed in a heated block to provide directional heat flux to promote capillary evaporation and preventing the scaffold temperature to fall into freezing regime. The heating method may be conductive or radiative in nature.

FIG. 7B illustrates one exemplary aspect of the present disclosure wherein additional heat source is provided from the inside of the cylinder. The heating method may be conductive or radiative in nature. The heat flux may be provided from one surface only or from both surfaces of the capillary substrate.

FIG. 8 shows vitrification of insulin at a concentration in a vitrification mixture (VM) of 120 IU/mL between a cartridge (FIG. 6B) and a flat capillary substrate (membrane) (FIG. 3). Each sample has 15 IU (125 μL VM). Six (6) “cartridge” samples and three (3) flat membrane samples vitrified together inside vacuum chamber (no heat). Moisture residual ratio (MRR) and protein recovery rate are evaluated after vitrification process and presented in the graph.

FIG. 9 at (A) shows the improved vitrification produced using a capillary substrate made of hydrophilic material. An originally hydrophobic membrane was treated with cold plasma to make it hydrophilic. Upon drug formulation suspension on the membrane, the liquid formed a nearly spherical droplet (top left) whereas the hydrophilic membrane allowed the liquid to flow into the capillary channels. During the vitrification process the liquid droplet on the hydrophobic membrane first boiled and then froze, whereas the liquid on the hydrophilic membrane vitrified quickly forming a glassy monolith. Upon the release of vacuum, the frozen droplet turned into liquid again, however the size was reduced to partial moisture loss. The efficacy of capillary assisted evaporation on vitrification is apparent utilizing the hydrophilic membrane. Photograph (B) of vitrification media applied to a circular scaffold (left) or applied to a solid glass substrate (right) and subjected to vacuum. The irregular appearance of the vitrification media placed on the glass surface is due to the rapid formation and rupture of bubbles and is evidence of boiling. Bubbles were not observed with the scaffold.

FIG. 10 shows the results of vitrification of insulin prepared on a membrane within a cylindrical 5 ml chamber with no applied heat illustrating that MRR is dramatically affected by biological material loading under these conditions.

FIG. 11 shows the weight loss recorded from insulin vitrification as set forth in FIG. 10.

FIG. 12 shows the MMR for vitrified insulin as described in FIG. 10, but with heat applied to the walls of the cylinder to provide heat energy to the sample being vitrified.

FIG. 13 shows the water loss seen in the vitrified insulin described in FIG. 12.

FIG. 14 shows successful reconstitution and retained functionality of vitrified mRNA from a low starting volume. (A) shows an agarose gel depicting liquid mRNA (lanes 2 and 3), reconstituted mRNA (lanes 4 and 5) and mRNA not vitrified by subjected to the same storage conditions (lanes 5 and 6). (B) shows expressed green fluorescent protein (GFP) following transfection with fresh mRNA (top), non-vitrified mRNA (middle) and reconstituted vitrified mRNA (bottom). (C) shows the percent of fluorescence of GFP relative to the positive control. The vitrification process did not negatively impact the amount of mRNA recovered or the functionality thereof.

FIG. 15 shows retained quantity and functionality of low starting mass of antibodies. Reconstituted alkaline-phosphatase (ALP) conjugated IgG provided a comparable response as a detection antibody (Ab) in an ELISA as fresh non-vitrified antibody. The vitrification process did not alter functionality, even when stored at 55° C., whereas 55° C. on non-vitrified antibody dramatically reduced functionality.

FIG. 16 shows a comparison between reconstituted co-vitrified luciferase and lucifernin and fresh non-vitrified luciferase and luciferin. The vitrification process did not negatively impact functional activity or luminescence as ATP was added.

FIG. 17 at (A) shows an ELISA wherein the antigen, capture antibody, and detection antibody were all vitrified and (B) shows activity where vitrified capture antibody and detection antibody are assayed with fresh antigen. Both (A) and (B) show a comparisons to an ELISA wherein all three components were non-vitrified as a control. The vitrification process did not alter antibody or antigen functional activity in the assay.

FIG. 18 shows an ELISA comparing vitrification on four components in an ELISA with non-vitrified fresh reagents. The vitrification process did not alter any components' ability to function properly in the assay.

FIG. 19 shows a 1× vitrification media, which contains 600 mM Trehalose, 5% Glycerol (with respect to weight of Trehalose) and remaining PBS is prepared and added to 1 inch diameter 8 μm PES membrane for vitrification. In 4 vitrification cycles, the temperature profile of two samples are recorded and in another vitrification cycle, the temperature profile of one sample is recorded. In total, the temperature data for 9 samples (inter-, intra-run) are recorded and plotted with error bars present. The data shows the consistency, robustness and repeatability of the process.

### DETAILED DESCRIPTION

As required, detailed aspects of the present disclosure are disclosed herein; however, it is to be understood that the disclosed aspects are merely exemplary of the disclosure that may be embodied in various and alternative forms.

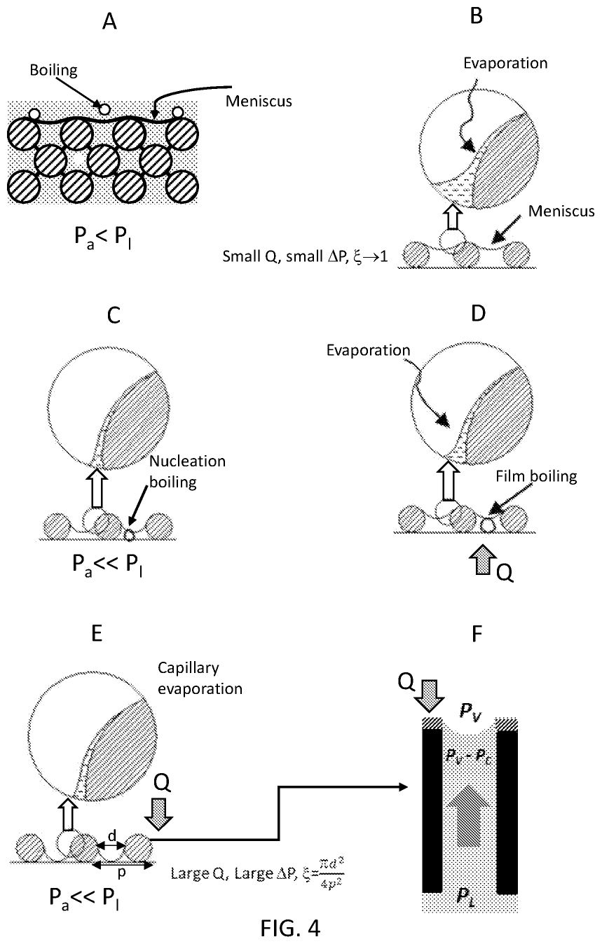

The present disclosure concerns methods for vitrification of biological samples and/or products that retain biological activity following long term storage at non-cryogenic temperatures. The methods of the present disclosure concern, in some aspects, providing a controlled temperature and a controlled atmosphere to the biological sample during desiccation and vitrification. In certain aspects, the present disclosure concerns applying a lower atmospheric pressure to a biological sample while providing heat energy to the same to prevent the sample from experiencing a freezing condition or boiling. As demonstrated in FIG. 4, the application of low atmospheric pressure can result in a significant drop in the temperature of the biological sample, providing conditions for ice crystal formation (e.g. freezing condition) within the biological sample. Accordingly, it is a further aspect of the present disclosure to provide heat to the biological sample to prevent the sample from experiencing a freezing condition and to prevent crystal formation within the biological sample during desiccation.

The following terms or phrases used herein have the exemplary meanings listed below in connection with at least one aspect:
As used herein “cryogenic” temperature or temperatures for “cryogenesis” or similar refer to a temperature at which a biological sample is exposed to freezing conditions. It will be understood in some aspects that the cryogenic temperature may include a freezing temperature of the biological sample and/or vitrification medium. It should further be understood that a cryogenic temperature is not bound by a particular threshold or range of values of temperatures in either Fahrenheit or Celsius, but instead can be determined by the relationship between temperature, pressure and molecular energy for the vitrification mixture of interest. It is further to be understood that as used herein, while certainly possible within the definition as set forth, “cryogenesis” and similar derivatives thereof are not limited to temperatures associated with liquid nitrogen at 1 atm or of about −80° C.
“Above cryogenic temperature,” as used herein, accordingly refers to a temperature above the freezing point of a vitrification mixture. A point “above cryogenic temperature” may further include temperature values wherein relation to the surrounding atmosphere and the molecular energy, a freezing condition is absent. Room temperature, as used herein, refers to a temperature of about 25° C.
As used herein, “boiling” may refer to a point at which a material transitions to a vapor, often marked by the formation of vapor bubbles within the material that can escape into a surrounding atmosphere and dissipate therein.
“Glass transition temperature” means the temperature above which material behaves like liquid and below which material behaves in a manner similar to that of a solid phase and enters into amorphous/glassy state. This is not a fixed point in temperature, but is instead variable dependent on characteristics of the vitrification mixture of interest. In some aspects, glassy state may refer to the state the vitrification mixture enters upon dropping below its glass transition temperature.
“Amorphous” or “glass” refers to a non-crystalline material in which there is no long-range order of the positions of the atoms referring to an order parameter of 0.3 or less. Solidification of a vitreous solid occurs at the glass transition temperature T_{g}. In some aspects, the vitrification medium may be an amorphous material.
“Crystal” means a three-dimensional atomic, ionic, or molecular structure consisting of one specific orderly geometrical array, periodically repeated and termed lattice or unit cell.
“Crystalline” means that form of a substance that is comprised of constituents arranged in an ordered structure at the atomic level, as opposed to glassy or amorphous. Solidification of a crystalline solid occurs at the crystallization temperature Tc.
“Vitrification” as used herein, is a process of converting a material into an amorphous material. The amorphous solid may be free of any crystalline structure.
“Vitrification mixture” as used herein, means a heterogeneous mixture of biological material(s) and a vitrification medium containing vitrification agents and optionally other materials.
“Biological material” or “biological sample” as used herein, refers to materials that may be isolated or derived from living organisms. Examples of biological materials include, but are not limited to, proteins, cells, tissues, organs, cell-based constructs, or combinations thereof. In some aspects, biological material may refer to mammalian cells. In other aspects, biological material may refer to human mesenchymal stem cells, murine fibroblast cells, blood platelets, bacteria, viruses, mammalian cell membranes, liposomes, enzymes or active fragments thereof, or combinations thereof. In other aspects, biological material may refer to reproductive cells including sperm cells, spermatocytes, oocytes, ovum, blastocysts, embryos, germinal vesicles, or combinations thereof. In other aspects, biological material may refer to whole blood, red blood cells, white blood cells, platelets, viruses, bacteria, algae, fungi, or combinations thereof
“Vitrification agent” as used herein, is a material that forms an amorphous structure, or that suppress the formation of crystals in other material(s), as the mixture of the vitrification agent and other material(s) cools or desiccates. The vitrification agent(s) may also provide osmotic protection or otherwise enable cell survival during dehydration. In some aspects, the vitrification agent(s) may be any water soluble solution that yields a suitable amorphous structure for storage of biological materials. In other aspects, the vitrification agent may be imbibed within a cell, tissue, or organ.
“Storable or storage” as used herein, refers to a biological material's ability to be preserved and remain viable for use at a later time.
“Hydrophilic” as used herein, means attracting or associating preferentially with water molecules. Hydrophilic materials with a special affinity for water, maximize contact with water and have smaller contact angles with water relative to hydrophobic materials.
“Hydrophobic” as used herein, means lacking affinity for water. Materials that are hydrophobic naturally repel water, causing droplets to form, and have large contact angles with water.
“Capillary” as used herein, pertains to or occurring in or as if in a tube of fine bore having a cross sectional area of about 2000 μm² or less.
“Cryopreservation” typically refers to rapid cooling of a biological sample, often through the use of liquid nitrogen due to its low temperature which will rapidly cool a liquid material, or small volume of biological materials by direct immersion. The rate of cooling reduces the mobility of the material's molecules before they can pack into a more thermodynamically favorable crystalline state. Over a more prolonged period, the molecules can arrange to crystallize which can produce damaging results, particularly in biological samples. Water is a significant concern in biological samples as it can crystallize quickly, and its abundance in living tissues can prove to be significantly damaging the more it is allowed to crystallize. Protective additives, often referred to as cryoprotectants, that interfere with the primary constituent's ability to crystallize may produce amorphous/vitrified material.

Provided are methods of preparing a vitrified biological sample that has robust stability when stored above a cryogenic temperature as well as kits, assays, and materials that may be produced thereby. Illustratively, a process for vitrification of one or more biological materials above cryogenic temperature is provided that includes: overlaying a vitrification mixture comprising a biological sample and a vitrification medium on a substrate comprising a capillary network, said substrate in a desiccation chamber; lowering the atmospheric pressure within the desiccation chamber; providing a heat energy from the surface to the vitrification mixture, wherein the heat energy is sufficient to prevent the vitrification mixture from experiencing a freezing condition; and desiccating the vitrification mixture by capillary action until the vitrification mixture enters a glassy state. The use of tailored temperature and pressure during vitrification enhances vitrification and stability of biological materials while reducing or eliminating the risk of exposure to freezing conditions or boiling, either of which could reduce the viability of the biological material(s).

In some aspects, the present disclosure concerns the vitrification of a vitrification mixture of a biological sample and a vitrification medium while avoiding exposure to a freezing condition and/or crystallization. A biological sample may include a cell, a collection of cells, a tissue sample, a cell fragment, an isolated and/or recombinant protein, an isolated or synthetic nucleic acid sequence (including RNA, DNA, single stands and double strands thereof), an expression vector, a bodily fluid, a hormone, a steroid, a cell receptor, a virion, a prokaryote, a simple eukaryote, a phospholipid, and/or a cell organelle.

In certain aspects, the present disclosure concerns vitrification of a biological sample with a vitrification medium that can include a glass forming agent. The identification of glass forming agents have opened opportunities for successful preservation of tissue. In the presence of appropriate glass forming agents, it is possible to store biological materials in a vitrified matrix above cryogenic temperatures with vitrification achieved by dehydration. Some animals and numerous plants are capable of surviving complete dehydration. This ability to survive in a dry state (anhydrobiosis) depends on several complex intracellular physiochemical and genetic mechanisms. Among these mechanisms is the intracellular accumulation of sugars (e.g., saccharides, disaccharides, oligosaccharides) that act as a protectant during desiccation. Trehalose is one example of a disaccharide naturally produced in desiccation tolerant organisms.

Sugars like trehalose may offer protection to desiccation tolerant organisms in several different ways. A trehalose molecule may effectively replace a hydrogen-bounded water molecule from the surface of a folded protein without changing its conformational geometry and folding due to the unique placement of the hydroxyl groups on a trehalose molecule. A sugar molecule may also prevent cytoplasmic leakage during rehydration by binding with the phospholipid heads of the lipid bilayer. Furthermore, many sugars have a high glass transition temperature, allowing them to form an above cryogenic temperature or a room temperature glass at low water content. The highly viscous ‘glassy’ state reduces the molecular mobility, which in turn prevents degradative biochemical reactions that lead to deterioration of cell function and death. Vitrification of biological materials by dehydration in the presence of glass forming sugar trehalose has been disclosed (see, N Chakraborty, et al., Biopreservation and Biobanking, 2010, 8 (2), 107-114).

The present disclosure concerns a vitrification process that combines low atmospheric pressure and heat energy to achieve even and rapid vitrification of a biological sample in a vitrification mixture. In some aspects, the present disclosure concerns application of heat energy to a vitrification mixture whereby vitrification occurs under reduced atmospheric pressure. In some aspects, heat energy is applied to a vitrification mixture to prevent the crystallization of the vitrification mixture or exposure to a freezing condition.

In further aspects, the temperature of the vitrification mixture is controlled during desiccation and/or vitrification. For example, a vitrification mixture is placed within a desiccation chamber and heat energy is applied to the vitrification mixture to restrict or prevent the vitrification mixture from experiencing a freezing condition. In some aspects, heat energy is transferred to the vitrification mixture to prevent crystallization therein.

In some aspects, the temperature of the biological sample is controlled within an applied vacuum or reduction in atmospheric pressure around the vitrification mixture. As is discussed herein, application of a low atmospheric pressure can significantly lower the temperature of the vitrification mixture causing the vitrification mixture to crystallize and/or go into cryogenesis. If the biological sample enters a freezing condition, irrevocable damage can occur therein which can negatively impact any desired activity or use when reconstituted. As is also identified herein, reduction in atmospheric pressure around the vitrification mixture can alter the molecular activity within the vitrification mixture, such that the boiling point is reduced. Similar to freezing, boiling the biological sample and/or vitrification medium or overheating can be detrimental. Boiling of a vitrification mixture can lead to biological sample loss of tertiary structure, crosslinking and degradation of the components therein, including proteins, fatty acids and nucleic acids and the like, rendering any activity upon reconstitution compromised. In certain aspects, the processes of the present disclosure concern maintaining a vitrification mixture at a temperature above a cryogenic temperature while in low atmospheric pressure such as a vacuum, partial vacuum or in a generally reduced pressure atmosphere.

In certain aspects, the vitrification mixture including the biological sample and the vitrification medium may be heated directly to control the temperature of such during desiccation. In other aspects, the vitrification mixture including the biological sample and the vitrification medium may have the temperature of such controlled by conduction, convection and/or radiation means. In other aspects, the vitrification mixture including the biological sample and the vitrification medium may have its temperature controlled by controlling the temperature outside of the desiccation chamber and relying on conduction through the desiccation chamber or portion thereof to control the temperature of the vitrification mixture. In such instances, it will be appreciated that the physical properties of the walls of the desiccation chamber will need to be taken into consideration. For example, a poorly thermal conducting material of the desiccation chamber may require an applied temperature different from that required by the vitrification mixture in order to allow the vitrification mixture to receive the appropriate heat energy. Such necessary adaptations will be readily appreciated by those in the art. In some aspects, heat may be applied through a heating pad, a heated bath, a flame, a heated bed, such as glass bead, a heated block and similar. In some cases, the heat energy may be from an electric source of generated heat and/or a heat energy released by combustion and/or a heat energy generated by electrical resistance.

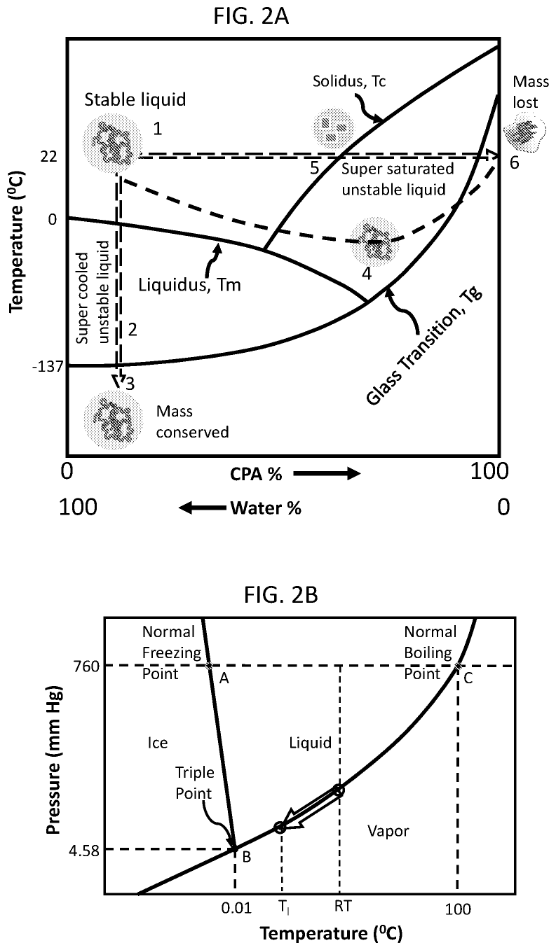

In some aspects, heat energy may be provided to the vitrification mixture through an underlying support substrate. While a porous material of a contiguous capillary network may also provide heat energy to the vitrification mixture, in some instances the porous material is of a poor thermally conducting material, such as glass or a polymer. However, the underlying substrate may be of a metal or similarly efficient conducting material and easily connected to a heat source outside of the desiccation chamber or an electrical source and provide heat by resistance created therein. The application of heat energy from the solid support may further provide a temperature gradient to assist in capillary evaporation (see, e.g., FIG. 2A).

In some aspects, the vitrification mixture including the biological sample and the vitrification medium is maintained at a temperature above its cryogenic temperature during vitrification under low atmospheric pressure. In some aspects, the vitrification mixture is preheated prior to desiccation under low atmospheric pressure. In other aspects, the vitrification mixture is heated during vitrification under low atmospheric pressure. In other aspects, heat is applied at or around the time vitrification commences. It will be appreciated that the amount of heat energy applied to the vitrification mixture may be constant or may vary during vitrification under low atmospheric pressure process. In some aspects, the introduction of low atmospheric pressure within the desiccation chamber can cause a rapid drop in temperature of the vitrification mixture. In such aspects, having the vitrification mixture ready to receive or already receiving heat energy can increase the recovery rate from the drop in temperature (see, e.g., FIG. 4).

In certain aspects, a constant temperature is applied to the vitrification mixture, such that the vitrification mixture is maintained at a temperature of from about T_{g} of the vitrification mixture in ° C. to about 40° C., including about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, and 39° C. In certain aspects, a higher temperature may be applied to the desiccation chamber or the porous material to provide the necessary heat energy to the vitrification mixture. Such applied temperatures may illustratively be of from about 15° C. to about 70° C., depending on the size of the desiccation chamber and the conductive means available to transfer effectively to the biological sample and/or vitrification medium.

In some aspects, the present disclosure concerns vitrification of a biological sample in low atmospheric pressure. In some aspects, the desiccation may occur in a desiccation chamber, whereby the vitrification mixture may be placed therein so as to be exposed to low atmospheric pressure. Such a desiccation chamber may be connected to a vacuum source to apply a low atmospheric pressure to the biological sample. As set forth herein, a vitrification mixture can be prepared with a vitrification medium or a cryopreservative such as trehalose and subjected to low atmospheric pressure, such as through application of a vacuum. In some aspects, the low atmospheric pressure is of from about 0.9 atm to about 0.005 atm, including 0.85, 0.8, 0.75, 0.7, 0.65, 0.6, 0.55, 0.5, 0.45, 0.4, 0.35, 0.3, 0.255, 0.25, 0245, 0.24, 0.235, 0.23, 0.225, 0.22, 0.215, 0.21, 0.205, 0.2, 0.195, 0.19, 0.185, 0.18, 0.175, 0.17, 0.165, 0.16, 0.155, 0.15, 0.145, 0.14, 0.135, 0.13, 0.125, 0.12, 0.115, 0.11, 0.105, 0.1, 0.095, 0.09, 0.085, 0.08, 0.075, 0.07, 0.065, 0.06, 0.055, 0.05, 0.045, 0.04, 0.035, 0.03, 0.025, 0.02, 0.015, and 0.01 atm.

In other aspects, the pressure within the desiccation chamber is lowered to a point above the triple point of the vitrification mixture. In other aspects, the pressure is lowered to a point above the triple point of water, such as greater than 0.006 atm. As set forth herein, lowered atmospheric pressure lowers the temperature of the vitrification mixture while also reducing its boiling point. In some aspects, the pressure within the desiccation chamber is lowered to about 0.04 atm or about 29 mmHg.

In some aspects of the present disclosure, the vitrification mixture is placed in a vacuum or partial vacuum at an elevated temperature or maintained at a temperature above the cryogenic temperature of the vitrification mixture at the atmospheric pressure applied, such that the vitrification mixture does not experience a freezing condition during the rapid decrease in atmospheric pressure. In further aspects, the temperature of the vitrification mixture will fall below the T_{g} of the vitrification medium to allow the vitrification of the biological sample.

In some instances, maintaining the low atmospheric pressure can require containing the vitrification mixture in a sealed enclosure, such as a desiccation chamber. It will be appreciated by those skilled in the art that providing and/or maintaining a low atmospheric pressure around the vitrification mixture will typically require that the desiccation chamber be capable of withstanding the low pressure therein. Such can be of any suitable or desired shape and/or material, being constrained by a requirement to maintain a low atmospheric pressure therein, requiring a sufficient seal and sufficient wall strength. The desiccation chamber can be operably connected to a vacuum source to lower the atmospheric pressure therein, while further allowing air to return upon vitrification completion. The desiccation chamber may be sufficiently sealed or closed so as to allow for an applied vacuum to effectively lower the atmospheric pressure in the desiccation chamber to the desired range.

In some aspects, the vitrification mixture is placed on or in a capillary network to enhance vitrification of the vitrification mixture. In further aspects, a capillary network can prevent the vitrification mixture from boiling under a reduced atmospheric pressure. The principles of capillary assisted evaporation are described in U.S. Pat. No. 10,568,318, which is incorporated by reference in its entirety herein.

In some aspects, the capillary network is of sufficient thickness to restrict liquid or fluid from accumulating on the surface thereof. To realize the capillary effect the liquid must be accommodated within the pores of the capillary substrate forming a meniscus. The liquid fraction (ξ) at the capillary interface, i.e., the volume occupied by the liquid, is a parameter for consideration for capillary evaporation. Capillary driven evaporation occurs when the viscous pressure drop in the liquid surpasses the maximum capillary pressure at the liquid-vapor interface. The liquid fraction is related to the overall pressure drop from the bulk to the liquid-vapor interface. Under atmospheric pressure and no applied heat flux (FIG. 4B) the liquid covers large area, leading to a liquid fraction, ξ→1. Under these conditions the capillary driven evaporation rate is minimal. Reducing the ambient pressure as shown in FIG. 4C, reduces ξ and in turn increases the evaporation rate. However, beyond certain threshold pressure drop, nucleation boiling can occur which is undesirable. An applied heat flux Q as shown in FIG. 4D can also enhance the evaporation rate, but the risk of film boiling exists, which is also undesirable. Applying the heat flux from the surface of the capillary meniscus as shown in FIG. 4E, eliminates the risk of film boiling. Under large ΔP and Q applied in a counter gradient fashion as shown in FIG. 4F, leads to the liquid meniscus confined to the pores, i.e., the liquid fraction ξ<<1 (˜0.25), resulting in highest evaporation rate while avoiding boiling

$$( ξ = \frac{π {d}^{2}}{4 {p}^{2}} ,$$

where p is distance between ridges or height of the capillary substrate and d the diameter of the circle formed by the shape of the liquid meniscus). Therefore, maintaining a temperature gradient between the surface and the bulk liquid leads to capillary evaporation as illustrated in FIG. 4F, where the fast evaporation can be achieved. As the liquid level recedes into the capillary substrate, capillary evaporation phenomena is still realized as long as the pressure gradient and temperature gradients are maintained. In some aspects, a capillary network under the mRNA or compositions thereof may assist in the evaporative processes during desiccation.

In some aspects, the heat energy is applied to a vitrification mixture as it undergoes vitrification on a capillary network. In some aspects, an underlying capillary network can allow for even and complete vitrification of a vitrification mixture receiving heat energy while protecting the vitrification mixture from boiling. The capillary network can be a contiguous network of capillaries meaning that there is no branching between a first end of a capillary and a second end of a capillary. In some instances, the capillary substrate network can be provided by an underlying porous material, such as a membrane, or an underlying contoured or ridged surface wherein the troughs and peaks thereof provide a bed sufficient to subject a liquid vitrification mixture to capillary action during vitrification.

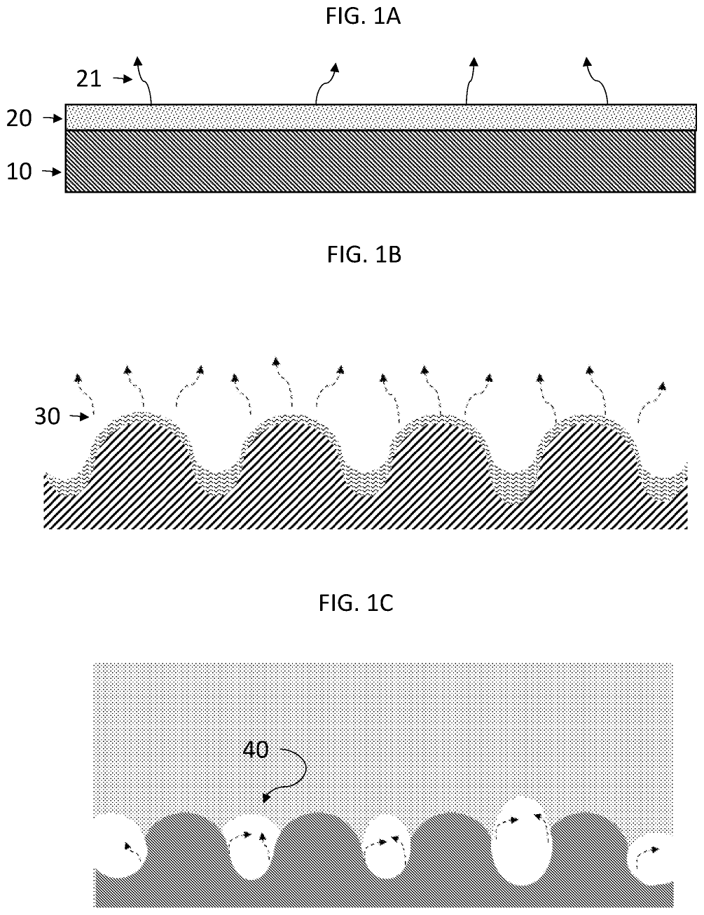

With reference to FIG. 1A, depicted is a contiguous hydrophilic bed 10 covered by application of a thin liquid layer of vitrification mixture 20. In some aspects, a protective fluid layer of vitrification mixture may protect the biological sample from boiling during exposure to low atmospheric pressure. Prevention of boiling under reduced atmosphere can be avoided and/or reduced with an extremely thin liquid film on a hydrophilic surface as shown in FIG. 1A. However, while prevention of boiling is possible, due to the limitation in the thickness of the liquid the available surface area reduces the amount of liquid that can be vitrified. The presence of a contoured surface, such as that set forth in FIG. 1B, effectively provides a surface upon which the vitrification mixture can be subjected to a capillary action due to preferential desiccation occurring at the peaks thereby drawing moisture up during the vitrification process and that can similarly protect the biological sample (that sits predominantly on the apices of the contours or ridges) from boiling and allows for significantly large sample volumes to be vitrified. Further, as the sample vitrifies at the peaks of the contours, capillary action draws fluid from the underlying trough, thereby promoting excellent vitrification and desiccation of the vitrification mixture. Similarly, if a porous material of a membrane of capillaries supports the biological sample within the capillaries, capillary action will draw fluid from the capillary channels during the vitrification process and provide even and complete vitrification and desiccation of larger volumes of the biological sample. However, as set forth in FIG. 1C, if the capillary action cannot successfully draw fluid up, such as in the case of a fluid loading that is too great, the liquid fills the surface patterns or is retained in the troughs, where bubble nucleation and boiling becomes dominant under reduced pressure which may lead to damage of sensitive molecules contained therein.

FIG. 2A is an overview of an aspect of the vitrification process of the present disclosure. Traditional vitrification is demonstrated in the pathway 1-2-3 where fast cooling a liquid (containing biological or other materials) to below the glass transition bypasses the freezing zone. The total mass of the material is conserved through the process. Cryogenic vitrification of a large amount material can be challenging due to heat transfer limitations and hence is generally carried out in vials that provide significant surface/volume ratio. Vitrification of materials can also be achieved by desiccation (bypassing the crystallization process), seen in pathway 1-5-6. In this aspect, significant mass loss (primarily water) occurs. Traditional dehydration approaches for biological materials have centered on establishing a sessile droplet on a substrate and evaporatively desiccating in a low humidity enclosure. The process is marked by a slow pace and uneven desiccation. A glassy skin forms at the interface between the liquid and vapor as the biological material therein desiccates. The formation of the glassy skin slows and ultimately prevents further desiccation of the biological sample, thereby limiting the biological sample to only a certain level of dryness with significant spatial non-uniformity of water content across the sample. As a result, some regions are not vitrified but will now degrade due to retained high molecular mobility. The desiccation rate can be facilitated by a large surface to volume ratio and specifically at reduced pressure.

In some aspects, the present disclosure concerns pathway 1-4-6 of FIG. 2A, where maintaining a desired temperature of the vitrification mixture and low pressure offer a hybrid of near cryogenic temperature and desiccation. However, with lower pressure, the boiling point is reduced. As shown in FIG. 2B, keeping the low pressure above the triple point of water can provide a temperature window between freezing and boiling for vitrification of the vitrification mixture. In some aspects of the present disclosure, the applied temperature maintains the temperature above the cryogenic point of the vitrification mixture at the low applied pressure. As further depicted in FIG. 2 and FIG. 4, the reduction in temperature from the applied low ambient pressure allows the temperature of the vitrification mixture to fall below the glass transition temperature without boiling, providing for even vitrification throughout the vitrification mixture.

FIG. 4A shows how fast desiccation of larger volume of liquid can be conveniently achieved under vacuum by deploying a porous material of a network of capillaries to facilitate capillary evaporation, such as through the introduction of a substrate of contiguous capillary channels. When the liquid accumulates on the surface of the capillary substrate, however, boiling still can occur in the accumulated liquid which is as described herein can be undesirable. The presence of a temperature gradient between the surface and the bulk liquid allows for capillary evaporation as illustrated in the FIGS. 4E and 4F, where the fast evaporation can be achieved.

Accordingly, in some aspects of the present disclosure, the volume of fluid present in the vitrification mixture can be established such that fluid can fill the capillary network without overflowing or pooling on the surface.

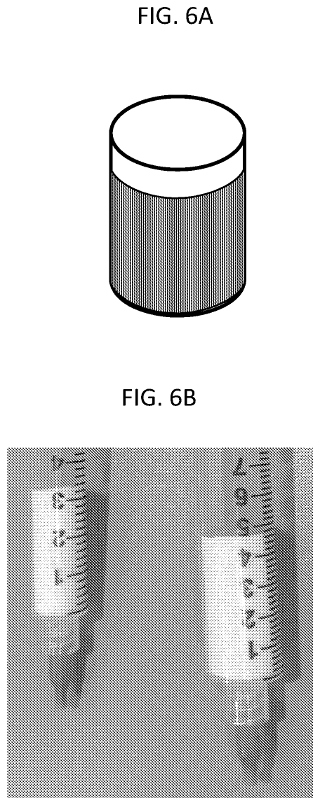

In certain aspects, the desiccation chamber or capillary substrate contained therein may be suitably patterned such that the walls of the chamber or capillary substrate provide a capillary network to the vitrification mixture when placed therein. For example, a contour or ridge such as that depicted in FIG. 1B can line the walls of a chamber (see, e.g., FIG. 6A) to provide an underlying capillary network. In other aspects, a porous material of a contiguous capillary network with a vitrification mixture therein is provided into a sealable desiccation chamber (see, e.g., FIG. 6B). In certain aspects, a porous material may include a membrane of a plurality of contiguous capillary channels.

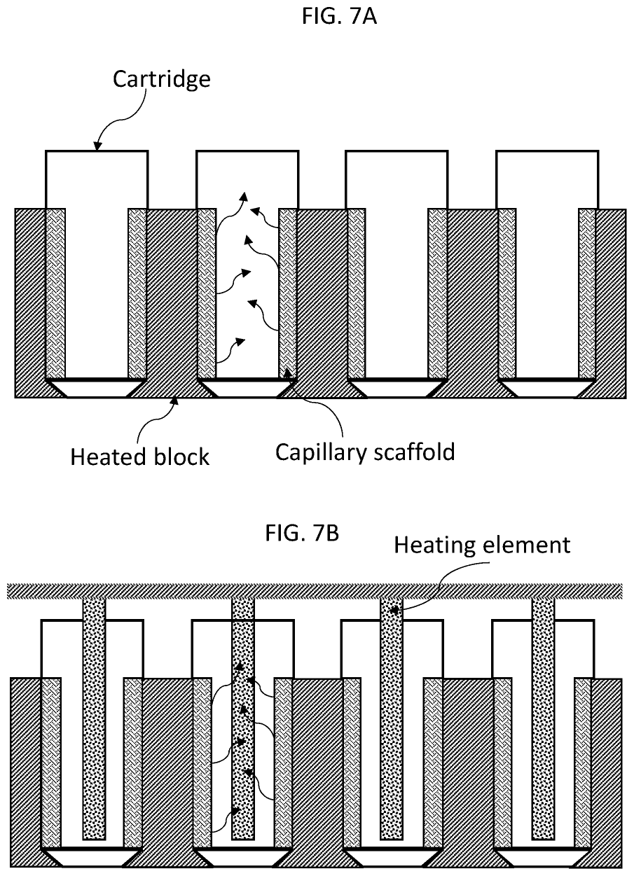

FIG. 7A illustrates placing a capillary substrate in a support that may itself be heated to avoid the vitrification mixture from experiencing a temperature below the T_{g}. In some aspects, a support scaffold may be included between the capillary substrate and the heating block or side of any chamber in which the capillary substrate is located to separate the capillary substrate from the surface from which heat is produced. This prevents direct heating from beneath the sample to substantially allow for desiccation from two directions or avoiding the greater heat exposed to the troughs of the membrane material, in some aspects.

As illustrated in FIG. 7B, a heating element may be instead or in addition introduced within the chamber allowing for directed heat application to the surface of the vitrification capillary substrate thereby promoting effective vitrification at the desired locations within/on the membrane to promote excellent capillary action and prevent boiling of the vitrification medium during vitrification.

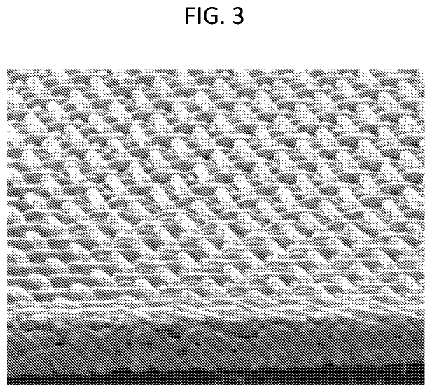

As illustrated in FIG. 1, the presence of contours and/or ridges provides capillary ridges to enhance vitrification. The presence of a vitrification mixture over the surface allows for fast evaporation from the peaks and by drawing the vitrification mixture toward the peaks by capillary action. The presence of a contiguous capillary network further allows the fluid volume of the vitrification mixture to evenly evaporate and prevent boiling while also preventing excess fluid build-up, which can also experience damaging boiling. Similarly, a porous material such as a membrane or substrate of contiguous capillary channels, may provide an underlying capillary network (see, e.g., FIG. 3). In such aspects, a porous material, such as a membrane or substrate, is housing the vitrification mixture and the capillary action therein provides for enhanced vitrification. Accordingly, in some aspects of the present disclosure, the vitrification mixture is placed on a contiguous capillary network. In further aspects, the vitrification mixture is placed on a patterned and/or ridged and/or contoured optionally porous material. In further aspects, the contiguous capillary network is formed by patterns and/or ridges and/or contours within walls of the desiccation chamber. In other aspects, the capillary network is provided by a porous material including a plurality of contiguous capillary channels.

The desiccation chamber should further be capable of or arranged to house the vitrification mixture therein. In some aspects, the desiccation chamber should be capable of housing the vitrification mixture on a porous material and/or a supporting substrate. In some aspects, the vitrification mixture is prepared for vitrification by placement upon a substrate. In some aspects, the substrate may be a porous material, such as a membrane and/or a bed of arranged capillaries. In further aspects, the walls of the desiccation chamber serve as a supporting substrate and are ridged and/or patterned and/or contoured to provide a capillary network therein.

In further aspects, a supporting substrate may be utilized to provide and/or transfer heat energy to the vitrification mixture. It will be appreciated by those skilled in the art that to provide heat energy effectively to the vitrification mixture, the supporting substrate may in some aspects be of a good conducting material, such as a metal. In other aspects, a porous material to provide a contiguous network of capillaries may be located between the vitrification mixture and the underlying solid support substrate.

As depicted in FIGS. 1 and 4, capillaries can provide an interface for rapid evaporation. The capillary network formed from either an underlying patterned ridged support or of a porous material such as a membrane may be made of a material that is not toxic and not reactive to the biomaterials or biological samples and does not react chemically or physically with the vitrification medium. The material can be of a suitable polymer, metal, ceramic, glass, or a combination thereof. In some aspects, a capillary network is formed from a material of polydimethylsiloxane (PDMS), polycarbonate, polyurethane, polyethersulphone (PES), polyester (e.g. polyethylene terephthalate), among others. Illustrative examples of a capillary channel containing membrane suitable as a surface in the devices and processes provided herein include hydrophilic filtration membranes such as those sold by EMD Millipore, Billerica, MA. In certain aspects, the porous material does not substantially bind, alter, or otherwise produce a chemical or physical association with a component of a biological sample and/or vitrification medium. The porous material is optionally not derivitized. Optionally, capillary channels may be formed in a substrate (e.g. desiccation chamber walls) of desired material and thickness by PDMS formation techniques, laser drilling, or other bore forming technique as is known in the art.

In some aspects, the capillary network is of sufficient thickness to restrict liquid or fluid from accumulating on the surface thereof. As depicted in FIG. 4A, increased fluid or liquid accumulation on the surface can lead to detrimental or damaging boiling. Increasing the thickness and/or layers of the capillary network can provide an increased space to handle increased fluid within the capillary channel or within the troughs. As set forth in FIG. 4E, the liquid fraction is determined by the relationship between the area of the circle formed by the meniscus and the height or distance between the ridges: ξ=πd²/4p². Optimally, ξ is of about 0.25, with an upper limit of about 1. At values below 0.25, the system may begin to dry up prior to desired vitrification.

In some aspects, a capillary network under the biological sample may assist in the evaporative processes during desiccation. As described herein, capillaries may be provided by patterning or contouring the walls of a desiccation chamber to effectively provide an underlying capillary bed or by providing a porous material of a contiguous capillary network, such as with a membrane. In some aspects, the capillary network provided by a porous material and/or a patterned and/or contoured surface features pores of about 20 μm or less, such that the pores provide underlying capillaries to assist in vitrification. In some aspects, the pores or peak to peak distance may be of an average opening of from about 20 μm to about 0.1 μm, including about 19, 18, 17, 16, 15, 14, 13, 12, 11, 10, 9, 8, 7, 6, 5, 4, 3, 2, 1.0, 0.9, 0.8, 0.7, 0.6, 0.5, 0.4, 0.3, and 0.2 μm. A capillary channel may have a length optionally defined by the thickness of a substrate that forms the channels or by one or a plurality of individual channels themselves. A capillary channel length is optionally about one millimeter or less, but is not to be interpreted as limited to such dimensions. Optionally, a capillary channel length is of about 0.1 microns to about 1000 microns, or any value or range therebetween. Optionally, a capillary channel length is of about 5 to about 100 microns, optionally of about 1 to about 200 microns, and/or optionally of about 1 to about 100 microns. A capillary channel length is optionally about 5, 10, 15, 20, 25, 30, 35, 40, 45, 50, 55, 60, 65, 70, 75, 80, 85, 90, 95, or 100 microns. In some aspects, the length of the capillary channels varies throughout a plurality of capillary channels, optionally in a non-uniform variation.

The cross-sectional area of the capillary channel(s) may be of about 2000 μm² or less. Optionally a cross-sectional area is of about 0.01 μm² to about 2000 μm², optionally of about 100 μm² to about 2000 μm², or any value or range therebetween. Optionally, a cross-sectional area of the capillary channel(s) is of about 100, 200, 300, 400, 500, 600, 700, 800, 900, 1000, 1100, 1200, 1300, 1400, 1500, 1600, 1700, 1800, 1900, or 2000 μm² or less.

Capillary assisted evaporation rate may be affected by both atmospheric demand (humidity, temperature and velocity of air/gas at the evaporating surface), and (i) the characteristics of the capillary channels that generate the driving capillary force, (ii) the liquid meniscus depth, and (iii) the viscous resistance to flow through the capillary. Consequently, complex and highly dynamic interactions between capillary properties, transport processes, and boundary conditions result in wide range of evaporation behaviors. For fast drying the key parameters may include: (1) the conditions that support formation and sustain a liquid network at the evaporating surface and (2) the characteristics that promote formation of capillary pressure that induce sufficient flow to supply water at the evaporating surface.

In some aspects, the porous material may be ridged and/or contoured or placed upon a ridged and/or contoured underlying support substrate, such that the porous material adopts a similar shape when placed or pressed thereon. As depicted in FIG. 1B, the contours and/or ridges of a patterned material may increase surface area to provide for increased exposure for evaporation.

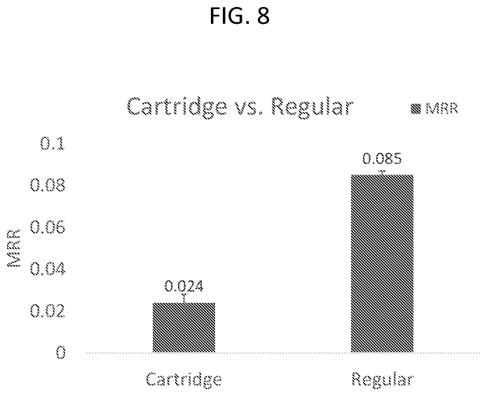

In further aspects, increased surface area of the porous material can be achieved by arranging or shaping a membrane. As depicted in FIG. 6A, a desiccation chamber with contoured walls may provide an increased surface area for the porous material. However, as depicted in FIG. 6B, shaping an otherwise flat porous material can further provide improved surface area for efficient capillary assisted vitrification. FIG. 8 sets forth a comparison between a flat membrane and a membrane curved within a cylindrical desiccation chamber. As seen in the resulting graph, the curving of the membrane within the desiccation chamber provided a lower moisture residual ratio than a flat membrane.

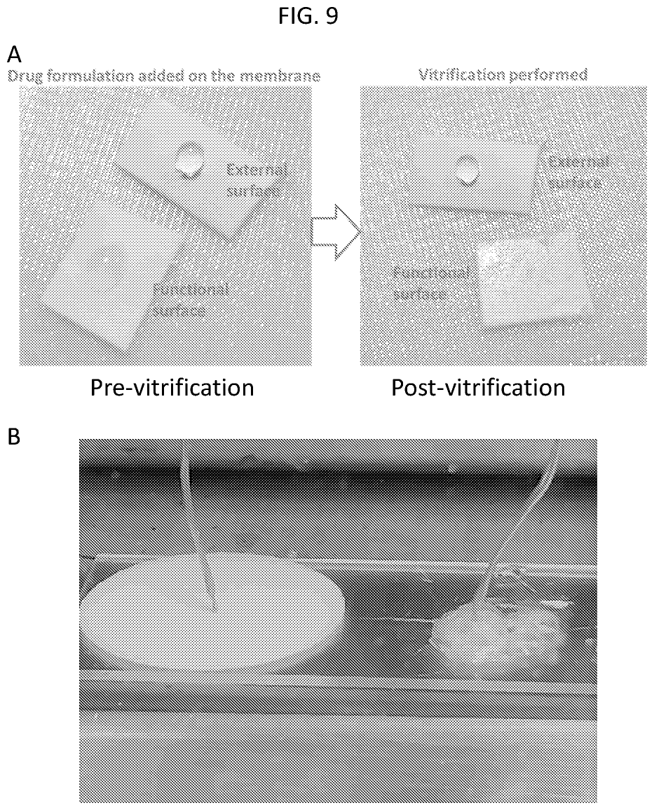

In some aspects, the capillary network is of a hydrophilic material. In other aspects, the capillary network may be of a hydrophobic material and further treated to be hydrophilic or more hydrophilic in nature, such as through exposure to a plasma. As depicted in FIG. 9, an originally hydrophobic membrane was treated with cold plasma to render it more hydrophilic. Upon drug formulation suspension on the membrane, the liquid formed a nearly spherical droplet (top left) whereas the hydrophilic membrane allowed the liquid to flow into the underlying capillary channels. During the vitrification process, the liquid droplet on the hydrophobic membrane first boiled and then froze, whereas the liquid on the hydrophilic membrane vitrified quickly forming a glassy monolith. Upon the release of vacuum, the frozen droplet turned into liquid again, however the size was reduced to partial moisture loss. The efficacy of capillary evaporation on vitrification is evident in the even vitrification seen with the hydrophilic membrane.

In some aspects, the biological sample is placed or covered or mixed in a vitrification medium to form a vitrification mixture. The presence of appropriate vitrification agents in a vitrification medium can be essential as the biological sample desiccates under the surrounding conditions as set forth herein. Fast desiccation methods as set forth herein by itself does not necessarily assure success in the viability of the cells or other vitrified biological material following desiccation. A vitrification medium that forms glass and/or that suppresses the formation of crystals in other materials may be required. A vitrification medium may also provide osmotic protection or otherwise enable cell survival during dehydration of the biological sample. Illustrative examples of agents to include in a vitrification medium may include one or more of the following: dimethylsulfoxide, glycerol, sugars, polyalcohols, methylamines, betines, antifreeze proteins, synthetic anti-nucleating agents, polyvinyl alcohol, cyclohexanetriols, cyclohexanediols, inorganic salts, organic salts, ionic liquids, or combinations thereof. In some aspects, a vitrification medium optionally contains 1, 2, 3, 4, or more vitrification agents.

In some aspects, a vitrification medium may include a vitrification agent at a concentration that is dependent on the identity of the vitrification agent. Optionally, the concentration of the vitrification agent is at a concentration that is below that which will be toxic to the biological sample being vitrified where toxic is such that functional or biological viability is not achieved upon subsequent sample use. The concentration of a vitrification agent is optionally of about 500 μM to about 6 M, or any value or range therebetween, including about 1, 2, 3, 4, or 5 M. For the vitrification agent trehalose, the concentration is optionally of about 1 M to about 6 M, including 2, 3, 4, or 5 M. Optionally, the total concentration of all vitrification agents when combined is optionally of about 1M to about 6M, including 2, 3, 4, or 5 M.

Trehalose, a glass forming sugar, has been employed in anhydrous vitrification and may provide desiccation tolerance in several ways. However, vitrified 1.8 moles/liter (M) trehalose in water has a glass transition temperature of −15 to 43° C. To achieve vitrification above 0° C., higher concentrations (6-8 M) are required which could be damaging to the biological materials. Alternatively, the vitrification medium may include buffering agents and/or salts to increase the T_{g} value of the VM. In some aspects, a vitrification medium may optionally include water or a solvent and/or a buffering agent and/or one or more salts and/or other components. A buffering agent may be any agent with a pKa of about 6 to about 8.5 at 25° C. Illustrative examples of buffering agents may include HEPES, TRIS, PIPES, MOPS, among others. A buffering agent may be provided at a concentration suitable to stabilize the pH of the vitrification medium to a desired level.

A vitrified medium including 1.8 M trehalose, 20 mM HEPES, 120 mM ChCl, and 60 mM betine provides a glass transition temperature of +9° C. An exemplary vitrification medium for the capillary assisted vitrification method disclosed herein may include trehalose, and one or more buffering agents containing large organic ions (>120 kDa) such as choline or betine or HEPES as well as buffering agent(s) containing small ions such as k or Na or Cl.

In some aspects, the biological sample is coated or immersed in a vitrification medium and placed on a support substrate to retain the biological sample during the steps of vitrification as set forth herein. In certain aspects, the capillary network absorbs some of the vitrification mixture while allowing a thin layer of fluid to remain.

In some aspects, the present disclosure concerns methods for vitrifying at least one biological sample, optionally 2, 3, 4, or more biological samples or materials. The methods include preparing a vitrification mixture through the assembly of the parts described herein. For example, a vitrification mixture of a biological sample and a vitrification medium is placed on or in contact with a solid support substrate. In some aspects, the underlying solid support is contoured and/or ridged to provide an underlying capillary network. In some aspects, the underlying support is part of a desiccation chamber, such as a wall thereof. In other aspects, a porous membrane can be placed between the vitrification mixture and a solid support. In some aspects, a contiguous capillary network supports the vitrification mixture and draws in fluid therefrom. The capillary network and/or porous material is to be of a sufficient thickness or quantity so as to avoid the presence and/or pooling of liquid above the surface of the capillary network.

The methods of vitrification of the present disclosure further include placing the vitrification mixture containing the biological sample in a desiccation chamber, the desiccation chamber being operably connected to a vacuum or other means for reducing the atmospheric pressure therein. In certain aspects, the biological sample is held in place on a porous or contoured material within the desiccation chamber. In some aspects, the biological sample is placed on part of the desiccation chamber, wherein the part is patterned and/or contoured so as to providing an underlying capillary network. In some aspects, a solid support substrate, a porous material, such as a membrane, and the biological sample are placed in the desiccation chamber.

The biological sample may optionally be coated and/or mixed with a vitrification medium in the desiccation chamber. In other aspects, the biological sample may be prepared with a vitrification medium prior to placement within the desiccation chamber.

Once assembled, the methods of the present disclosure may include reducing atmospheric pressure around the vitrification mixture, providing capillary-assisted evaporation to the vitrification mixture and/or applying heat energy to the vitrification mixture/capillary network without inducing boiling therein. As described herein application of all three can provide for rapid and even vitrification and desiccation of the vitrification mixture, while avoiding experiencing a cryogenic temperature and avoiding boiling, thereby significantly reducing any damage to the biological sample during the process.

In certain aspects, the methods include exposing the biological sample to a low or reduced atmospheric pressure. In some aspects, the exposure to low atmospheric pressure occurs by operating a vacuum connected to the desiccation chamber. In some aspects, the atmospheric pressure is lowered to a point above the triple point of the biological sample and/or vitrification medium. In other aspects, the pressure is lowered to about 0.04 atm or 29.5 mmHg.

In some aspects, the methods include applying heat energy to the biological sample during exposure to the low atmospheric pressure. In some aspects, a source of heat energy is also operably connected to the desiccation chamber or placed therein. In some aspects, the source of heat energy is applied to an underlying solid support beneath a porous material and/or capillary substrate. It will be appreciated that the source of heat energy is operably placed in some aspects to provide heat to the vitrification mixture from one or both ends of a capillary or from the direction of peaks in an undulating vitrification membrane surface. In further aspects, the heat energy source provides a temperature gradient across the capillary substrate and/or porous membrane to assist in evaporation. In further aspects, the heat energy may be applied to the exterior of the desiccation chamber.

Once the vitrification mixture is placed within the desiccation chamber, the atmospheric pressure therein is lowered. In some aspects, the atmospheric pressure is lowered to a point above the triple point of the biological sample therein. In other aspects, the atmospheric pressure is lowered to a point above the triple point of water. In further aspects, the pressure is lowered within the desiccation chamber to about 0.04 atm.

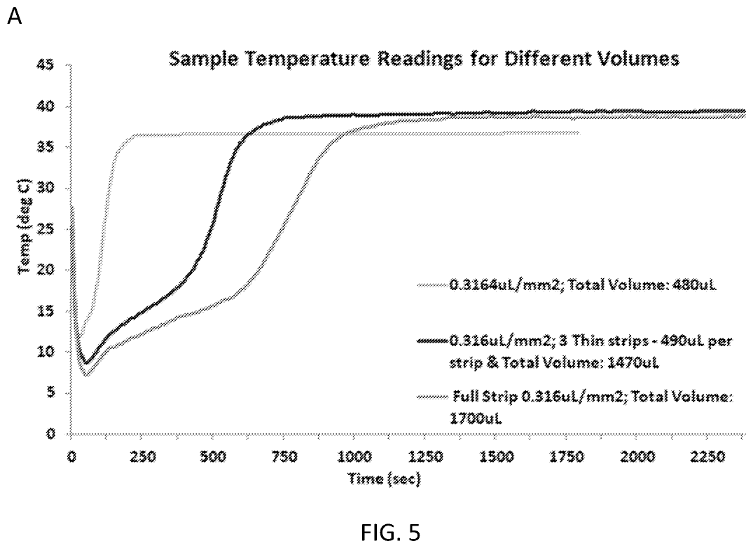

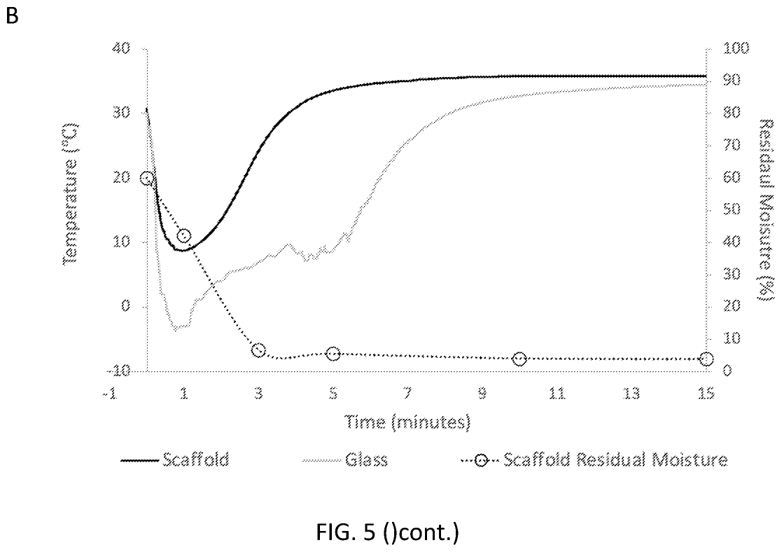

FIG. 5 depicts results seen from applying 37° C. heat from a wire mesh as the underlying solid support and glass membranes thereon as the capillary substrate. FIG. 5 shows a comparison between the membrane and volume size and the rate at which the biological sample temperature recovers following lowered pressure when liquid loading is maintained constant. As set forth in FIG. 5, in all cases, application of the vacuum leads to a rapid drop of temperature of the vitrification mixture, yet the smaller membranes produced faster complete vitrification as observed by return to the starting temperature. With further reference to FIG. 5, it is seen that the temperature of the biological sample plateaus once vitrification is complete.

In some aspects, the methods of the present disclosure include providing capillary assisted evaporation of a vitrification mixture. In some aspects, the underlying capillary substrate provided by a contoured and/or ridged support or by a porous membrane will provide the necessary features required to enhance evaporation.

In some aspects, the methods of the present disclosure may be performed for a desiccation time. A desiccation time is a time sufficient to promote suitable drying to vitrify the vitrification medium. A desiccation time is optionally from about 1 second to about 1 hour, including but not exceeding about 10 s, 30 s, 1 min, 5 min, 10 min, 20 min, 25 min, 30 min, 35 min, 40 min, 45 min, 50 min and 55 min. Optionally, a desiccation time is of from about 1 second to about 30 min, optionally of from about 5 seconds to about 10 min.

The duration a biological material may remain viable in vitrified state during storage above cryogenic temperature may vary from one sample material to the next. In some aspects, a biological material may remain viable while in storage above cryogenic temperature for 2-20 days. In other aspects, a biological material may remain viable while in storage above cryogenic temperature for 10 weeks. In other aspects, a biological material may remain viable in storage above cryogenic temperature for up to one year. In other aspects, a biological material may remain viable while in storage above cryogenic temperature for up to 10 years.

Alternatively, after vitrification above cryogenic temperatures employing the teachings and devices of the current disclosure, the vitrified biological material can be stored at cryogenic temperatures and/or in liquid nitrogen for very long periods without risk of crystal formation within the material. For many biological materials, this is a preferred approach to avoid cryoinjury that commonly occurs during direct vitrification at cryogenic temperatures. A preferred approach in one aspect is to vitrify the biological materials at room temperatures utilizing low concentrations of vitrification agents (e.g. <2 M trehalose) and then immediately store at cryogenic temperatures. Therefore, the said device is optionally made out of materials storable at a temperature between −196° C. to 60° C. following the vitrification according to the teachings of the current disclosure.

It is an aspect of the present disclosure that smaller volumes of a sample can also be successfully vitrified and provide full or exceptionally high performance upon reconstitution. In some aspects, a volume of 10 μL or less can be vitrified through the processes as set forth herein and retain full ability upon reconstitution. In some aspects, the processes of vitrification as set forth herein can vitrify samples of from about 0.1 μL to 10 μL or greater, including about 0.2, 0.3, 0.4, 0.5, 0.6, 0.7, 0.8, 0.9, 1, 2, 3, 4, 5, 6, 7, 8, and 9 μL of sample. For example, as set forth in the working examples herein, providing 3 μL of ribonucleic acids to the vitrification mixture and storing the resulting vitrified material for more than 3 months at temperatures well above room temperature resulted in less than 3 ng of lost material from a starting 3 μg when reconstituted. Further, despite ribonucleic acids being typically susceptible to damage and degradation, the vitrification processes herein successfully protected the small volume of ribonucleic acids such that upon reconstitution, retained over 75% transfection activity compared to a freshly transfected sample, identifying that the vitrification processes set forth herein can safely and effectively preserve small volumes of sample.

In some aspects, the smaller volume of the sample is with respect to the unit area occupied by the sample. In some aspects, the sample to be vitrified is provided at between about 0.10 μL/mm² and about 0.25 μL/mm², including about 0.11, 0.12, 0.13, 0.14, 0.15, 0.16, 0.17, 0.18, 0.19, 0.20, 0.21, 0.22, 0.23, and 0.24 μL/mm². Accordingly, a 10 μL sample would occupy between about 40 and 100 mm². In some aspects, the sample may cover an area of the substrate of from about 0.5 mm² to about 100 mm².

In some aspects, the present disclosure provides processes for vitrification that allow for excellent recovery of low massed samples. In some aspects, the processes herein allow for excellent recovery of samples with a mass of 5 μg or less, including about 4, 3, 2, and 1 μg of sample. In some aspects, the mass of the sample can be below 1 μg, including masses of 100 ng or less. In some aspects, the mass of the sample to be vitrified is of from about 0.1 to 1 μg, including 0.2, 0.3, 0.4, 0.5, 0.6, 0.7, 0.8, and 0.9 μg. For example, as set forth in the working examples herein, antibody fragments of less than 1 μg were vitrified according to the processes set forth herein, stored, and then reconstituted and assessed for activity. The vitrification process did not noticeably hinder or alter antibody activity in comparison to a non-vitrified fresh control in an ensuing ELISA, even with storage conditions ranging from −20° C. to 55° C.

In some aspects of the present disclosure, the processes of vitrification can be applied to more than one sample or material for successful storage and preservation. In some aspects, multiple samples can be vitrified within the same vitrification mixture. It will be apparent that some samples may require isolation from other materials prior to a desired end use. In such aspects, the separated materials can be vitrified in separate vitrification mixtures on the same capillary substrate, utilizing physical space to isolate. In other aspects, sequential vitrifications may be utilized such that one material is vitrified upon a second previously vitrified material and so on.

In some aspects, the present disclosure concerns two or more materials vitrified within the same area of a capillary substrate. Optionally, 2, 3, 4, 5, or more biological materials or reagents may be vitrified into a single capillary substrate. Such may be achieved by co-vitrification or by successive vitrification steps, or combinations thereof. In some aspects, one or more materials may be bound to the underlying capillary substrate, such as an immobilized biological material. For example, immobilization of an antibody on the capillary substrate provides a region within which the single use can occur, such that a detectable signal is provided on the capillary substrate. It will be apparent that one or more materials may be immobilized on the capillary substrate, as well as that not all materials need to be immobilized. For example, for an ELISA, one antibody may be immobilized and another may not be until performing the assay and links to the immobilized antibody.

In some aspects, the present disclosure provides for a capillary substrate with one or more vitrified materials required for a single use, such as an assay or a measurement utilizing materials vitrified therein. In some aspects, more than one capillary substrate or piece thereof may be utilized for each material for an assay. In some aspects, two or more materials for a single use may be vitrified together, either within the same area of the membrane or by being vitrified within the same vitrification medium or co-vitrified. It will be apparent that other aspects may include two or more materials vitrified within the same area of a capillary substrate and one or more materials for the assay are vitrified on a separate capillary substrate, either on an entirely different substrate or on a different area or region of the same substrate.

In some aspects, the present disclosure concerns multiple materials vitrified to allow for storage up to a point when all can be reconstituted for a single use, such as an assay. While the examples set forth herein demonstrate retained quantity and functionality of biological materials, it will be appreciated that other materials can similarly be vitrified without loss or alteration thereto.

In some aspects, the present disclosure concerns kits or assemblages of vitrified materials to allow or provide for a single use. In some aspects, such may include one or more vitrified biological materials. In some aspects, such may include one or more vitrified reagents for a single use. Reagents may include buffers, salts, substrates, enzyme substrates, antigens, chemical compounds, peptides, and small nucleic acids such as primers or siRNA. It will be apparent that in some aspects, a single use assemblage or kit may require or optionally include one or more non-vitrified materials, such as liquid solutions for reconstitution, non-perishable or oxidation-susceptible chemicals or salts, buffers, cell media, anti-microbiotics, or similar. It will further be appreciated that not all materials need to be vitrified for a kit for single use. For example, a user may supply a test material or sample with which the vitrified material(s) may be used for analysis or observation thereof.

In some aspects, the present disclosure concerns kits that include one or more materials vitrified by the processes as set forth herein. Such may include one or more vitrified biological samples, one or more vitrified reagents, and/or one or more accompanying solutions. In some aspects, the kit may include only one or more vitrified biological samples or only one or more vitrified reagents. In some aspects, the kit may include at least one vitrified biological sample and at least one vitrified reagent. In some aspects, the kit may include at least one biological sample co-vitrified with another biological sample and/or at least on reagent. In some aspects, all or some components of the kit may be vitrified independently, either on separate regions or areas of a shared underlying substrate or on individual substrates, or combinations thereof.

In some aspects, the kits may provide all or part of a single use application. Such applications may include materials for a polymerase chain reaction (PCR), an enzyme-linked immunosorbent assay (ELISA), cell culture, obtaining a medical sample, providing a medicament, providing a vaccine, a laboratory assay, an enzyme assay, cellular transfection, DNA mutagenesis or editing, expression of an exogenous protein or peptide, and similar. The kits may include one or more vitrified nucleic acids, proteins, peptides, polynucleotide chains, DNA, RNA, mRNA, tRNA, antibodies, buffers, salts, enzymes, enzyme-substrates, lipids, cationic lipids, ionizable lipids, sterols, steroids, therapeutics, adjuvants, carriers, excipients, substrates, catalysts, amino acids, deoxyribonucleotide triphosphate (dNTPs), combinations thereof or similar.

For example, as set forth herein, it is identified that vitrification of a capture antibody, an antigen, and a detection antibody does not adversely impact a resulting ELISA assay. Accordingly, a kit may include one or more of a vitrified capture antibody, an antigen, and/or a detection antibody. A kit may also include a vitrified detection reagent. A kit may include a reconstitution solution. A kit may further include reagents to allow for vitrification to allow for storage of a material, such as a vitrification medium. Similarly, for PCR, a kit may include one or more of vitrified primer(s), vitrified polymerase, vitrified reverse transcriptase, vitrified vector, vitrified template DNA/RNA, vitrified dNTPs, or similar. Some kits may provide a vitrified enzyme and/or a vitrified substrate to the enzyme.

### EXAMPLES

1. Sample Heating and Capillary Substrate Size

Initial examples were arranged to assess the role of applying heat to the desiccation process under low atmospheric pressure and the effect of capillary substrate size on the ability to rebound the sample temperature following application of the vacuum.

Glass membranes (Ahlsltrom 8951) of different shapes were loaded with a liquid containing 4 wt % BSA, 15 wt % Trehalose Dihydrate, 0.75 wt % Glycerol, 2 wt % Tween-20, and water. The liquid loading per mm² of the membrane was kept at 0.316 ml for all cases. For Case 1, the membranes were cut into 0.25 inch diameter circles and each was loaded with 10 ml liquid sample. A total of 48 membrane samples containing 480 ml were loaded on a heated (i.e. 37° C.) wire mesh inside a vacuum chamber. For Case 2, three long membrane strips (240 mm×6.23 mm) each containing 470 μl liquid were loaded on a heated wire mesh, also at 37° C. For case 3, a single strip (240 mm×22 mm) containing 1700 μl liquid was utilized. The chamber in each case was evacuated to 29.5 mmHg. The temperature-time plots indicate the liquid undergoing the desiccation process. FIG. 5 shows the sample temperature over the time course of the applied vacuum.

As seen in FIG. 5, each sample experienced sudden drop in temperature. The drop was managed to remain above cryogenic temperature by surface heating the sample through wire mesh. As the water loss occurred through desiccation process, the sample temperature rose until the majority of water was lost indicating near completion of the vitrification process. Although, the liquid loading per unit area was constant, the number and dimensions of the capillary substrates causing evaporation impacted the time taken for vitrification process. These data demonstrate that for larger volumes of material, more rapid vitrification can be achieved by using smaller membranes and dividing the vitrification medium accordingly.

2. Hydrophilic Capillary Substrates

An analysis of the suitability of hydrophilic and hydrophobic capillary systems was assessed by suspending a drug composition in a vitrification medium and depositing an equal volume of each on each type of membrane. An originally hydrophobic membrane was treated with cold plasma to make it hydrophilic. FIG. 9 illustrates the pre and post vitrification results. Upon drug formulation on the hydrophobic membrane, the applied liquid formed a nearly spherical droplet (top left) whereas the hydrophilic membrane allowed the liquid to flow into the underlying capillary channels. During the vitrification process, the liquid droplet on the hydrophobic membrane first boiled and then froze, whereas the liquid on the hydrophilic membrane vitrified quickly forming a glassy monolith. Upon the release of vacuum, the frozen droplet turned into liquid again, however the size was reduce to partial moisture loss. The efficacy of capillary evaporation on vitrification is evident in the presented even vitrification on the converted hydrophilic capillary membrane.

3. Desiccation Chamber

Instead of patterning the surface of the cartridge (FIG. 6A) with a capillary pattern (e.g. FIG. 1B), a capillary system can be created by providing a hydrophilic capillary substrate that lines the walls of a desiccation chamber (FIG. 6B). In the present example, a hollow syringe was utilized due to its ability to be sealed and operably connected to a vacuum source. The cylindrical nature further provided a straightforward shape to apply heat to the sample therein.

A hydrophilic membrane was curved and inserted inside the cylindrical body. Upon suspension of the vitrification solution, the liquid found its way into the capillary channels of the underlying membrane until the membrane was fully loaded with liquid.

For the assessment, a vitrification solution of insulin (Sigma Aldrich) was prepared at a concentration in VM of 120 IU/mL. Each sample had 15 IU (125 μL VM).

A total of six samples in a curved chamber and three flat membrane samples were initially vitrified together inside vacuum chamber with no applied heat. The moisture residual ratio (MRR) and protein recovery rate were evaluated after vitrification process (FIG. 8).

The mixing ratio of the biological sample to the aqueous medium was examined. Again insulin was utilized and mixed with PBS as set forth in Table 1:

| Insulin in VM | 0 IU/mL | | 10 IU/mL | | 50 IU/mL | | 100 IU/mL | | 200 IU/mL | |
|---|---|---|---|---|---|---|---|---|---|---|
| Sigma Stock | 0 | μL | 32.130 | μL | 160.648 | μL | 321.296 | μL | 642.592 | μL |
| VM | 238.4 | mg | 238.4 | mg | 238.4 | mg | 238.4 | mg | 238.4 | mg |
| PBS | 861.975 | μL | 829.846 | μL | 701.327 | μL | 540.679 | μL | 219.383 | μL |
| Total volume | 1000 | μL | 1000 | μL | 1000 | μL | 1000 | μL | 1000 | μL |
| Theoretical Dry | 225.295 | mg | 225.516 | mg | 226.399 | mg | 227.503 | mg | 229.710 | mg |
| Weight | | | | | | | | | | |

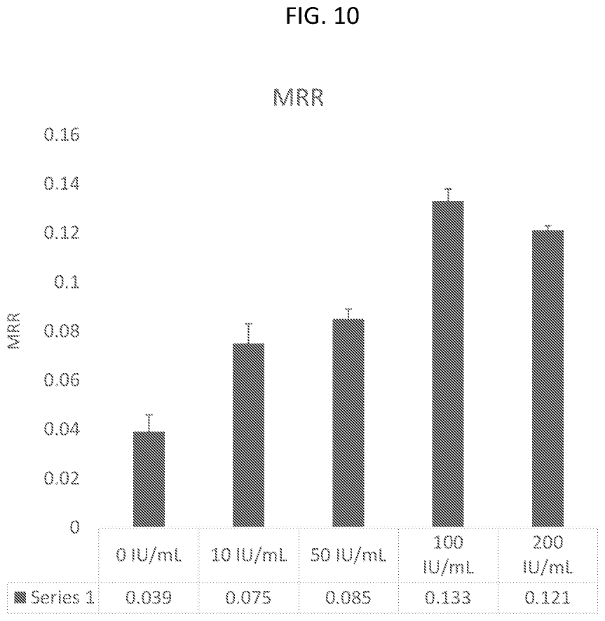

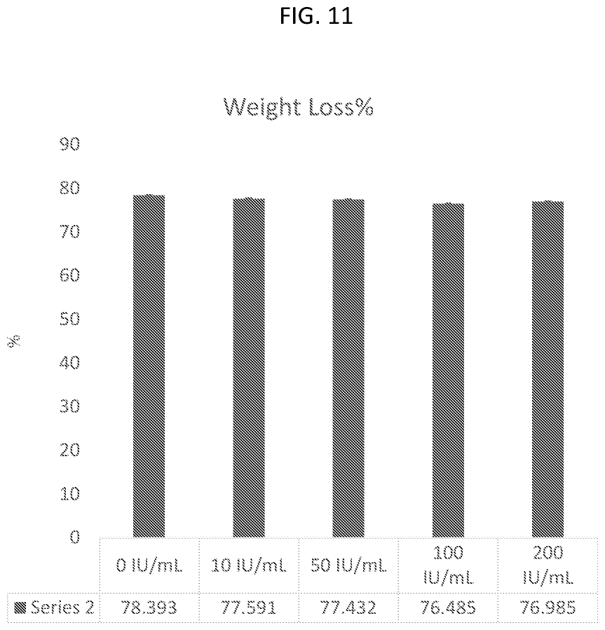

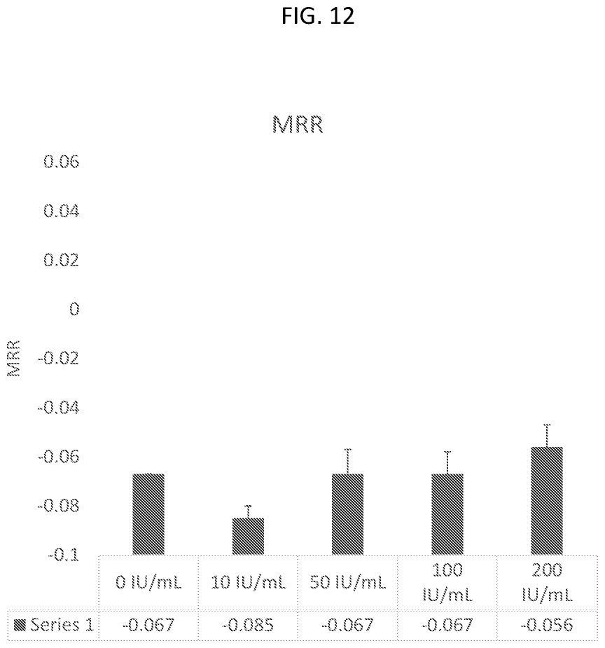

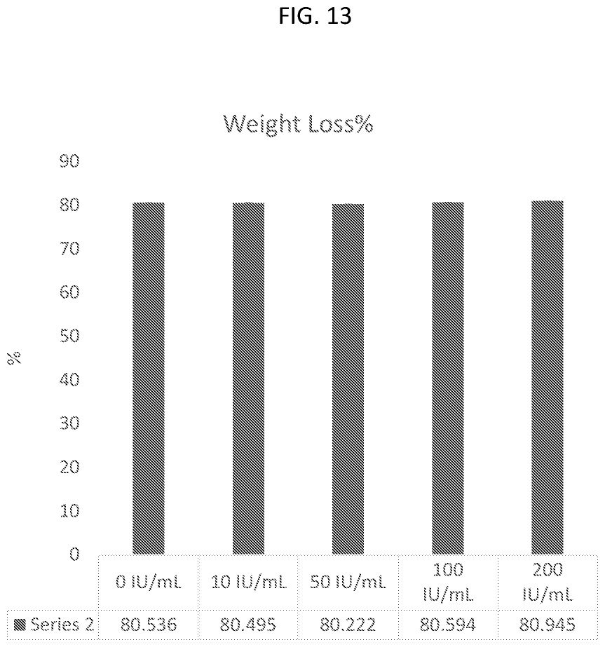

The vitrification process was performed in a vacuum for 30 mins in the presence of heat or no applied heat, with MRR and weight loss measured. FIGS. 10 and 11 show the results obtained with no heat applied and FIGS. 12 and 13 show results with heat applied during the vitrification process to maintain the sample above the cryogenic temperature and thereby prevent exposure to freezing conditions.

4. Small Sample Volume

To assess the ability to recover small samples from the vitrification process, small volumes of mRNA encoding green fluorescent protein (GFP) were utilized. For the vitrification process, an 8 μm PES membrane (capillary substrate) was first cut into ¼ inch diameter size and autoclaved. A 2× vitrification medium (VM) with contains 1200 mM (or 454 mg/mL) trehalose and 22.7 mg/mL Glycerol in PBS was prepared and then mixed with equal volumes of the mRNA (Dasher GFP mRNA, 3870FS Aldevron) stock. The mixture was allowed to incubate for 5 minutes before pipetting 6 μL to each vitrification capillary substrate. Following pipetting the solution on to the membrane, the samples were covered with a polymer lid and loaded into the vitrification chamber. For each vitrified sample, in total 6 μL was loaded to the membrane before vitrification, within which there was 3 μL of naked mRNA stock, which contains 3 μg of mRNA.

After vitrification, samples were sealed in mylar pouches and stored at 55° C. before testing.

One hundred days after vitrification storage at 55° C., the samples were reconstituted with 50 μL of Fluorobrite media with brief vortexing to release the mRNA. The mRNA was then quantitated using a Take3 plate on a BioTek Synergy H1 microplate reader. Table 2 shows the obtained quantifications.

TABLE 2

| | 260/280 | Concentration |
|---|---|---|
| Samples | ratio | (ng/μL) |
| Positive Control | 2.02 | 67.28 |
| (Fresh mRNA) | | |
| Vitrified mRNA on | 2.19 | 64.36 |
| PES membrane and | | |
| stored at 55° C. | | |
| for 100 days | | |

Portions of the mRNA were then used for transfection or for visualization on an agarose gel.

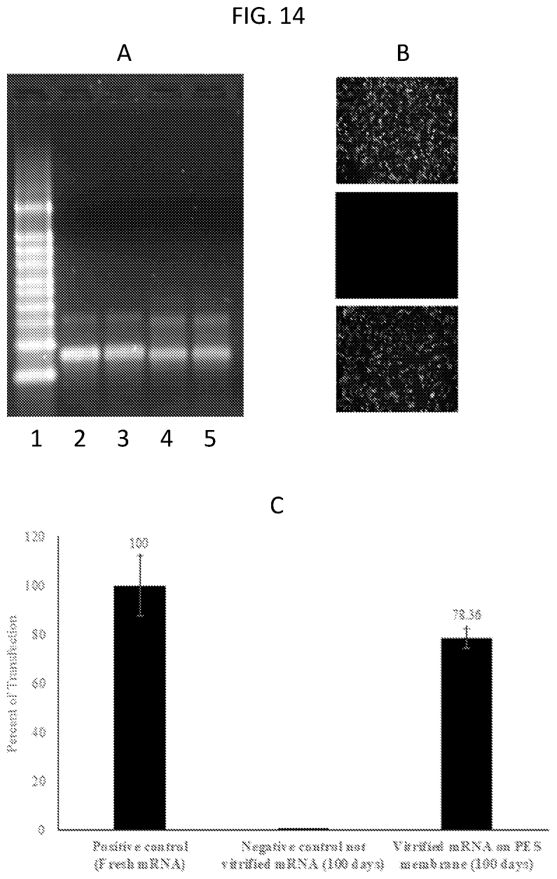

For the agarose gel, a ladder of 3 μL of Millennium™ RNA Markers (AM7150) with 3 μL of dye and 5 μL of water was used in the first lane of a 1.2% agarose gel. For a positive control, the stock mRNA was diluted to 125 ng/μL with 3 positive control: dilute mRNA stock to 125 ng/μL then 1 μL of the diluent stock was mixed with 3 μL of dye and 5 μL of water. For the vitrified samples 125 ng of reconstituted mRNA was mixed with 3 μL of dye and 5 μL of water. After running the gel at 85V for an hour, the gel was stained with SYBR Green II for 30 mins on a shaker read in a BioRad transilluminator. FIG. 14A shows a captured image of the gel with lanes 2 and 3 being fresh mRNA that was stored at −80° C., lanes 4 and 5 being reconstituted vitrified mRNA and 5 and 6 being non-vitrified mRNA stored at 55° C.

For transfection, a positive control of 4 μL lipofectamine (Lipofectamine™ MessengerMAX™ Transfection Reagent, Lipofectamine™ MessengerMAX™ Transfection Reagent) was added to 16 μL media, allowed to incubate for 10 minutes. In another tube, 1 μL of mRNA (fresh sample) was added to 19 μL of media and incubated for 10 mins. The two solutions were then mixed and incubated for another 5 minutes before transferring 10 μL cell plates seeded with 0.9×10⁶ cells/mL CHO (Chinese hamster ovary) cells. For a negative control and for the vitrified samples, after quantification the volume of mRNA was normalized to that required to make 1 μg of mRNA and added to lipofectamine after a 10-minute incubation. FIG. 14B shows collected images of GFP expression, with the top panel being the positive control of fresh mRNA, the middle being the negative control of non-vitrified mRNA stored at 55° C., and the bottom panel being reconstituted mRNA. FIG. 14C shows the obtained percentage of transfection efficiency relative to that obtained with the positive control.

The mRNA vitrified on the PES membrane and stored at 55° C. for 100 days maintains the mRNA integrity, purity, and stability similar to the fresh liquid mRNA that was stored at −80° C.

5. Low Mass Vitrification

The ability to vitrify smaller masses of sample and be able to reconstitute and maintain functionality was next assessed utilizing an alkaline phosphatase rabbit anti-human IgG. 1.2 μL of a stock of 0.6 mg/mL AffiniPure Rabbit Anti-Human IgG (H+L) (Cat: 309-055-082, Jackson ImmunoResearch Laboratories, Inc., West Grove, PA) was mixed with 36.3 μL PBS and then mix with another 37.5 μL of a prepared 2× vitrification medium of 1200 mM (or 454 mg/mL) trehalose and 22.7 mg/mL glycerol in PBS. 65 μL was withdrawn and aliquotted onto a 1 inch diameter, 8 μm pore size PES membrane. In total two samples are vitrified for 30 mins with a bed heating temperature set at 37° C.

After vitrification, one sample was stored at −20° C. and the other sample was stored at 55° C. A liquid control was made by mixing 4.8 μL of antibody stock with 255.2 μL of PBS and stored at 55° C.

The samples were reconstituted and assayed in an ELISA for retained presence and functionality of antibody. A capture antibody (AffiniPure Mouse Anti-Human IgG, F(ab′)₂ fragment specific, Jackson ImmunoResearch Laboratories, Cat 209-005-097) was diluted to 1 μg/mL and coated on an ELISA plate using 100 μL per well, which was allowed to incubate at RT for 1 hour, followed by washing the plate 3 times using 300 μL/well with assay diluent. The plate was then blocked with 200 μL/well assay diluent for 1 hour at RT, followed by washing the plate 3 times using 300 μL/well with assay diluent.

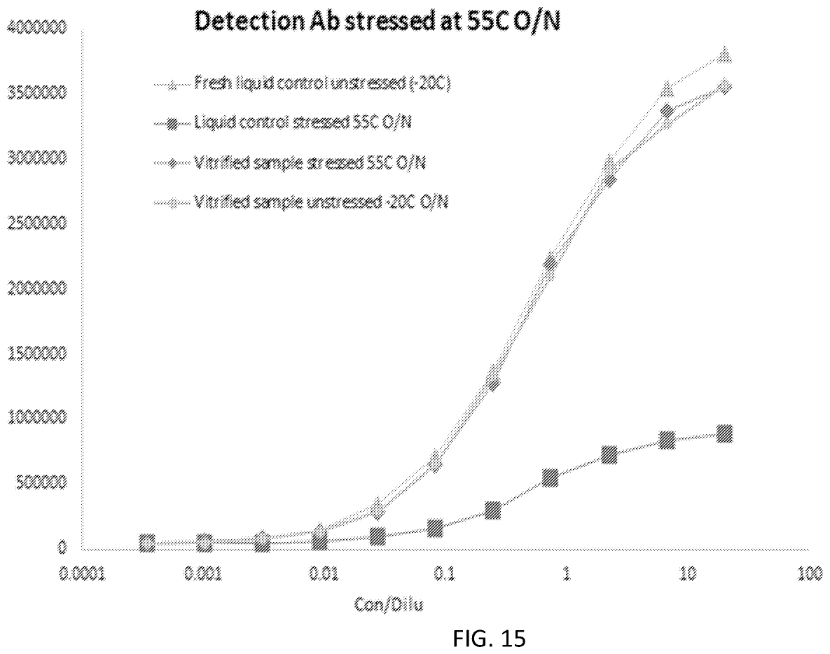

The intended antigen then underwent a 3-fold serial dilution (Human IgG whole molecule, Rockland, Cat 009-0102) on a dilution plate and 100 μL of the diluted antigen was transferred to the corresponding wells, incubated at RT for 1 hour, followed by washing the plate 3 times using 300 μL/well with assay diluent. 100 μL/well of the reconstituted ALP conjugated antibody was added to the plate and incubated at RT for 1 hr, followed by 3 times of washing with 300 μL/well of assay diluent. Next, 100 μL/well 4-MUP substrate solution was added and incubated 20 mins in dark at RT. The fluorescence signal was read from the plate using BioTek Synergy H1 microplate reader and the data then processed. FIG. 15 shows the relative fluorescence units (RFU) at each respective concentration/dilution assayed. 0.84 μg of vitrified alkaline-phosphatase detection antibody provided a similar ELISA response as the positive control, irrespective of being stored at −20° C. or 55° C., which indicates that the vitrified ALP conjugated detection antibody retains both the original quantity and functionality as prior to vitrification and storage. As also seen in FIG. 15, even a single overnight storage of the liquid antibody (i.e. not vitrified) demonstrated significant decline in functionality.

6. Multiple Sample Vitrification

It was next assessed the ability to retain quantity and functionality following co-vitrification of an enzyme and its chemical substrate. For this series of tests, a luciferase stock (QuantiLum® Recombinant Luciferase, Promeg, E170) and the luciferin substrate (Beetle Luciferin, Potassium Salt, Promega, E1601) were used. 2 μL of the luciferase stock and 55.6 μL of luciferin were mixed with 57.6 μL of a vitrification medium that contains 1200 mM trehalose and 22.7 mg/mL glycerol in PBS. The mixture was aliquotted on to a one inch diameter 8 μm PES membrane and allowed to vitrify for 30 minutes with a heat bed temperature of 37° C.

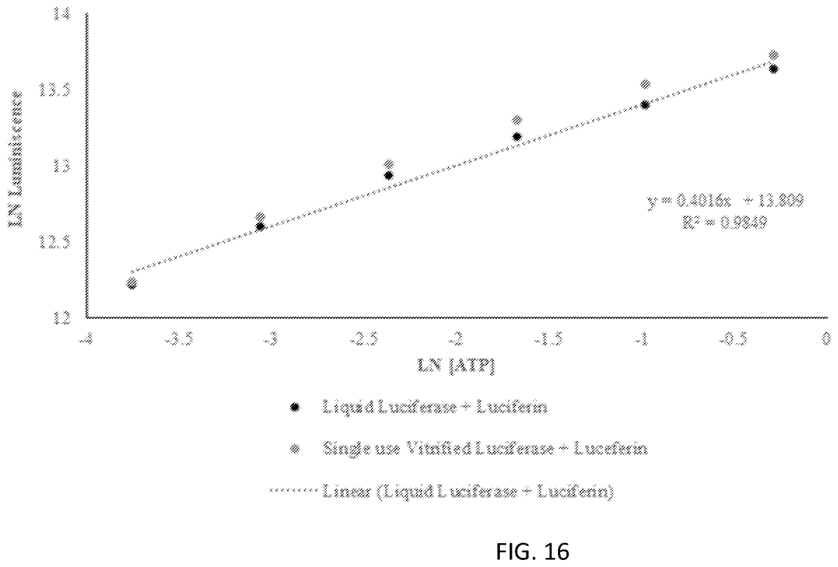

Following reconstitution, the vitrified luciferase/luciferin was compared to activity obtained with fresh luciferase and luciferin as ATP is added. An ATP standard was prepared from a 1 mM ATP solution with 2-fold serial dilution. 50 μL of the corresponding ATP was added to each well. For the fresh luciferase/luciferin, 2.3 μL of luciferase stock was mixed with 71.7 μL of luciferin stock and 1426 μL of 4.7 mM MgSO₄/EPPS, with 50 μL going to each well. For the vitrified samples, the reconstituted volumes were added with 1164.1 μL of 4.5 MgSO₄/EPPS, with 50 μL being used per well. FIG. 16 shows the comparison between the fresh and the vitrified samples. The single use luciferase and luciferin vitrified preparation shows a similar luminescence signal to freshly prepared liquid luciferase and luciferin, identifying that the functional activity and luminescence signal are not compromised by vitrification and non-cryogenic storage.

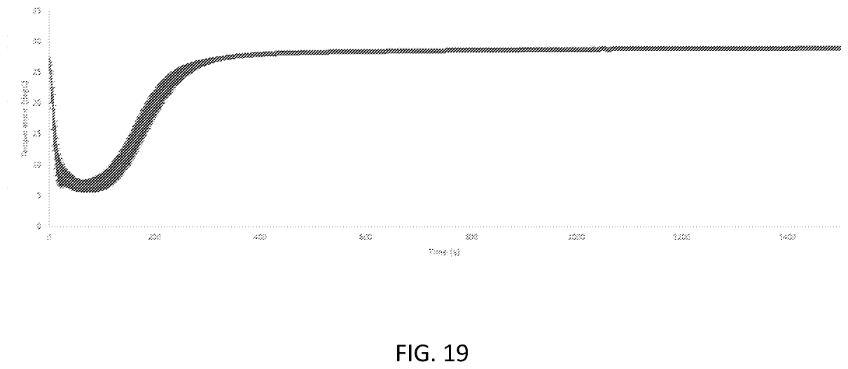

Additionally, as shown in FIG. 19, the vitrification process can be repeated multiple times. FIG. 19 shows a vitrification media (600 mM Trehalose, 5% Glycerol (with respect to weight of Trehalose) and PBS) added to a 1 inch diameter 8 μm PES membrane for vitrification. In 4 vitrification cycles, the temperature profile of two samples were recorded and in another vitrification cycle, the temperature profile of one sample is recorded. In total, the temperature data for 9 samples (inter-, intra-run) are recorded and plotted with error bars present. The data shows the consistency, robustness and repeatability of the process.

7. Individual Multi-Component Vitrification

It was next assessed whether isolated components for an assay could be individually vitrified and still retain quantity and functionality. Accordingly, an ELISA was performed with individually vitrified antigen, capture antibody and alkaline phosphatase conjugated antibody for detection. A vitrification medium containing 1200 mM trehalose and 22.7 mg/mL glycerol in PBS was used to individually vitrify: a capture AffiniPure Mouse Anti-Human IgG, F(ab′)₂ fragment specific (Jackson ImmunoResearch Laboratories, Cat 209-005-097) by combining 20 μL with 55 μL PBS and 75 μL vitrification medium; the antigen of whole human IgG (Human IgG whole molecule, Rockland, Cat 009-0102) by combining 12 μL with 63 μL PBS and 75 μL of the vitrification medium; and, the detection antibody (Alkaline Phosphatase AffiniPure Rabbit Anti-Human IgG (H+L), Jackson ImmunoResearch Laboratories, Inc., West Grove, PA, Cat 309-055-082) by combining 3.6 μL with 71.4 μL PBS and 75 μL of the vitrification medium. 50 μL of each mixture was applied to the membrane, and then vitrified for 30 mins with heat bed temperature set at 37° C.

Each of the vitrified reagents were reconstituted in the following volume of assay diluent before used in the corresponding ELISA steps: capture 12 mL, antigen 2 mL, detection 12 mL. A summary of the conditions is presented in Table 3.

TABLE 3

| | | | vitrification | | |
|---|---|---|---|---|---|
| | Stock | mass/vitrified | volume/vitrified | reconstitution | conc. after |
| Name | Conc. | sample | sample | volume | reconstitution |
| Mouse Anti- | 1.8 mg/mL | 12 μg | 50 μL | 12 mL | 1 μg/mL |
| Human IgG - | | | | | |
| capture | | | | | |
| Human IgG - | 10 mg/mL | 40 μg | | 2 mL | 20 μg/mL |
| antigen | | | | | |
| ALP | 0.6 mg/mL | 0.72 μg | | 12 mL | 0.06 μg/mL |
| conjugated | | | | | |
| Rabbit Anti- | | | | | |
| Human IgG | | | | | |

In the ELISA itself, for the fresh liquid control the capture antibody was diluted to 1 μg/mL and coated in the corresponding wells using 100 μL per well. For the vitrified capture antibody sample, the corresponding wells were coated with 100 μL per well. Both were incubated at RT for 1 hour, followed by washing the plate 3 times using 300 μL/well with assay diluent. The plate was then blocked with 200 μL/well assay diluent for 1 hour at RT, followed by washing the plate 3 times using 300 μL/well with assay diluent. A 3-fold serial dilution for both fresh antigen and vitrified antigen on a dilution plate was prepared and then 100 μL of the diluted antigen was transferred to the corresponding wells, incubated at RT for 1 hour, followed by washing the plate 3 times using 300 μL/well with assay diluent. Then, 100 μL/well of the fresh or vitrified ALP conjugated antibody was added to the plate and incubated at RT for 1 hr, followed by 3 times of washing with 300 μL/well of assay diluent. Then 100 μL/well of 4-MUP substrate solution was added and incubated 20 mins in dark at RT. The fluorescence signal as then read.

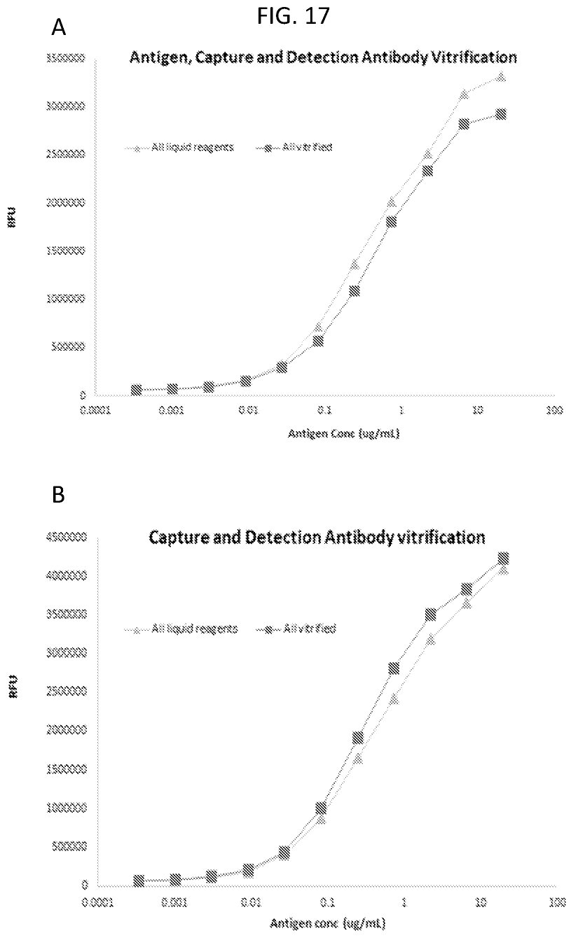

As seen in FIGS. 17A and 17B, vitrified ELISA antibody and/or antigen has comparable signal to that observed with the freshly prepared liquid, non-vitrified control. FIG. 17A shows the ELISA of vitrified antigen, capture and detection antibodies and FIG. 17B shows the vitrified capture and detection antibodies with a non-vitrified antigen. The vitrification process did not alter antibody or antigen functional activity in the assay.

A further vitrified component for a four-part assay was then assessed. Accordingly, the Staphylococcus enterotoxin B (SEB) was introduced as a further intermediary in an ELISA to assess for loss of quantity and/or function. Accordingly, the capture (Rabbit anti SEB polyclonal, Cat AB-R-SEB) stock was diluted to 560 μg/mL and then 10.8 μL was mixed with 14.2 μL PBS and 25 μL of the vitrification medium. The antigen (Staphylococcus enterotoxin B (SEB)) required 1.3 μL to be mixed with 23.7 μL PBS and 25 μL vitrification medium. The detection antibody (Mouse anti SEB mab, Cat AB-R-MAB) stock was from a concentration of 420 μg/mL using 5.8 μL with 19.2 μL PBS and 25 μL vitrification medium. The alkaline phosphatase (ALP) conjugate (Alkaline phosphatase conjugated Goat anti mouse IgG, Jackson ImmunoResearch Laboratories, 115-055-146) was mixed by combining 4.8 μL with 20.2 μL PBS and 25 μL of the vitrification medium. 50 μL of each was applied to the washed membrane, and then vitrified for 30 mins with heat bed temperature set at 37° C. Each of the vitrified reagents were reconstituted in the following volume of assay diluent before used in the corresponding ELISA steps: capture 12 mL, antigen 2 mL, detection 12 mL. Table 4 sets forth a summary of the volumes and concentrations utilized.

TABLE 4

| | Stock | | mass/vitrified | | vitrification | reconstitution | conc. after | |
|---|---|---|---|---|---|---|---|---|
| Name | conc. | | sample | | volume | volume | reconstitution | |
| Rabbit anti | 5.6 | mg/mL | 6 | μg | 50 μL | 12 mL | 0.5 | μg/mL |
| SEB - | | | | | | | | |
| capture | | | | | | | | |
| SEB - | 11.7 | mg/mL | 15 | μg | | 2 mL | 10 | μg/mL |
| antigen | | | | | | | | |
| Mouse anti | 4.2 | mg/mL | 2.4 | μg | | 12 mL | 0.2 | μg/mL |
| SEB - | | | | | | | | |
| detection | | | | | | | | |
| ALP | 0.6 | mg/mL | 2.88 | μg | | 12 mL | 0.24 | μg/mL |
| conjugate | | | | | | | | |
| GxM | | | | | | | | |

For the ELISA, as a fresh positive control the capture antibody was diluted to 0.5 μg/mL and coated on the corresponding wells using 100 μL per well. For the vitrified capture antibody sample, the corresponding wells were coated with 100 μL per well. The wells were incubated at RT for 1 hour, followed by washing the plate 3 times using 300 μL/well with assay diluent. The plate was then blocked with 200 μL/well assay diluent for 1 hour at RT, followed by washing the plate 3 times using 300 μL/well with assay diluent.

Next, a 2-fold serial dilution was performed for both fresh antigen and vitrified antigen from a starting concentration of 10 μg/mL on a dilution plate and then 100 μL of the diluted antigen was transferred to corresponding wells, incubated at RT for 1 hour, followed by washing the plate 3 times using 300 μL/well with assay diluent. Then, 100 μL/well of the fresh or vitrified detection antibody was added to the plate and incubated at RT for 1 hr, followed by 3 times of washing with 300 μL/well of assay diluent. Next, 100 μL/well of the fresh or vitrified ALP conjugated antibody was added to the plate and incubated at RT for 1 hr, followed by 2 times of washing with 300 μL/well of assay diluent. Finally, 100 μL/well of the 4-MUP substrate solution was added and incubated 20 mins in dark at RT. The fluorescence signal from the plate was read using BioTek Synergy H1 microplate reader and the data appropriately processed.

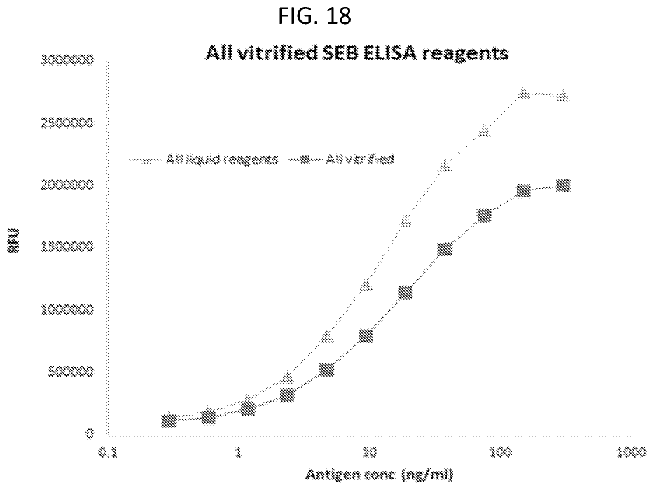

As seen in FIG. 18, vitrification of all four components in the assay still yielded comparable results to that observed in the freshly prepared liquid, non-vitrified control. The vitrification process, therefore, does not alter quantity or functionality for the antibodies or the SEB antigen.

### FURTHER EXAMPLES

A first aspect of the present disclosure, either alone or in combination with any other aspect herein, concerns a process for vitrification of one or more biological materials above cryogenic temperature, the process comprising: a) overlaying a vitrification mixture comprising a biological sample and a vitrification medium on a substrate comprising a capillary network, said substrate in a desiccation chamber; b) lowering the atmospheric pressure within the desiccation chamber; c) providing a heat energy from the surface to the vitrification mixture, wherein the heat energy is sufficient to prevent the vitrification mixture from experiencing a freezing condition; and d) desiccating the vitrification mixture by capillary action until the vitrification mixture enters a glassy state.

A second aspect of the present disclosure, either alone or in combination with any other aspect herein, concerns the process of the first aspect, wherein the capillary network is provided by contours along the surface of the substrate.

A third aspect of the present disclosure, either alone or in combination with any other aspect herein, concerns the process of the first aspect, wherein the substrate is a wall of the desiccation chamber or is associated with a wall of the desiccation chamber.

A fourth aspect of the present disclosure, either alone or in combination with any other aspect herein, concerns the process of the first aspect, wherein the capillary network within the desiccation chamber is contacted by an underlying solid support substrate.

A fifth aspect of the present disclosure, either alone or in combination with any other aspect herein, concerns the process of the first aspect, wherein vitrification of the vitrification mixture occurs in less than 30 minutes.

A sixth aspect of the present disclosure, either alone or in combination with any other aspect herein, concerns the process of the fifth aspect, wherein vitrification of the vitrification mixture occurs in less than 10 minutes.

A seventh aspect of the present disclosure, either alone or in combination with any other aspect herein, concerns the process of the first aspect, wherein the heat energy is provided by heating the vitrification mixture.

An eighth aspect of the present disclosure, either alone or in combination with any other aspect herein, concerns the process of the first aspect, wherein the atmospheric pressure is lowered to a value of from about 0.9 atm to about 0.005 atm.

A ninth aspect of the present disclosure, either alone or in combination with any other aspect herein, concerns the process of the eighth aspect, wherein the atmospheric pressure is lowered to about 0.004 atm.

A tenth aspect of the present disclosure, either alone or in combination with any other aspect herein, concerns the process of the first aspect, wherein the heat energy provided is sufficient to prevent crystallization within the vitrification mixture during vitrification.

An eleventh aspect of the present disclosure, either alone or in combination with any other aspect herein, concerns the process of the first aspect, wherein the provided heat energy is sufficient to keep the biological sample at a temperature of from about 0° C. to about 40° C. during said vitrifying.

A twelfth aspect of the present disclosure, either alone or in combination with any other aspect herein, concerns the process of the first aspect, wherein said vitrification medium comprises trehalose, glycerol and betine and/or choline.

A thirteenth aspect of the present disclosure, either alone or in combination with any other aspect herein, concerns the process of the first aspect, wherein the capillary network is hydrophilic.

A fourteenth aspect of the present disclosure, either alone or in combination with any other aspect herein, concerns the process of the first aspect, wherein the capillary network comprises contiguous capillary channels.

A fifteenth aspect of the present disclosure, either alone or in combination with any other aspect herein, concerns the process of the first aspect, wherein the biological sample is selected from the group consisting of a nucleic acid, an amino acid, a polynucleotide chain, a peptide, a protein, and an antibody.

A sixteenth aspect of the present disclosure, either alone or in combination with any other aspect herein, concerns the process of any one of the first through fifteenth aspects, wherein said vitrification mixture is overlayed on said substrate at a volume of 10 μL or less.

A seventeenth aspect of the present disclosure, either alone or in combination with any other aspect herein, concerns the process of the sixteenth aspect, wherein the total volume of biological sample is from 0.1 μL to 10 μL.

An eighteenth aspect of the present disclosure, either alone or in combination with any other aspect herein, concerns the process of any one of the first through fifteenth aspects wherein said biological sample is present is said vitrification mixture at 1 μg or less.

A nineteenth aspect of the present disclosure, either alone or in combination with any other aspect herein, concerns the process of the eighteenth aspect, wherein the biological sample is of a total mass of 5 μg or less, optionally less than 1 μg.

A twentieth aspect of the present disclosure, either alone or in combination with any other aspect herein, concerns the process of the nineteenth aspect, wherein the biological sample is of a total mass of 0.1 μg to 1 μg.

A twenty-first aspect of the present disclosure, either alone or in combination with any other aspect herein, concerns the process of any one of the first through fifteenth aspects, wherein the vitrification mixture further comprises a second material.

A twenty-second aspect of the present disclosure, either alone or in combination with any other aspect herein, concerns the process of the twenty-first aspect, wherein the second material is a reagent to the biological sample.

A twenty-third aspect of the present disclosure, either alone or in combination with any other aspect herein, concerns the process of any one of the first through fifteenth aspects, wherein the vitrification mixture further comprises a first reagent material.

A twenty-fourth aspect of the present disclosure, either alone or in combination with any other aspect herein, concerns the process of the twenty-third aspect, wherein the vitrification mixture further comprises a second reagent material.

A twenty-fifth aspect of the present disclosure, either alone or in combination with any other aspect herein, concerns the process of the twenty-fourth aspect, wherein the first reagent material comprises an enzyme or active fragment thereof.

A twenty-sixth aspect of the present disclosure, either alone or in combination with any other aspect herein, concerns the process of the twenty-fifth aspect, wherein the second reagent material comprises a chemical compound that undergoes an enzyme-catalyzed reaction with the enzyme or active fragment thereof.

A twenty-seventh aspect of the present disclosure, either alone or in combination with any other aspect herein, concerns the process of the twenty-fourth aspect, wherein the first reagent material comprises a first antibody or active fragment thereof.

A twenty-eighth aspect of the present disclosure, either alone or in combination with any other aspect herein, concerns the process of the twenty-seventh aspect, wherein the second reagent material comprises an antigen or a second antibody or active fragment thereof, wherein the second antibody or active fragment thereof binds to a different epitope from the first antibody or active fragment thereof.

A twenty-ninth aspect of the present disclosure, either alone or in combination with any other aspect herein, concerns the process of the twenty-fourth aspect, wherein the first reagent material comprises a polynucleotide chain.

A thirtieth aspect of the present disclosure, either alone or in combination with any other aspect herein, concerns the process of the twenty-ninth aspect, wherein the second reagent material comprises deoxyribonucleotide triphosphate (dNTPs).

A thirty-first aspect of the present disclosure, either alone or in combination with any other aspect herein, concerns the process of the twenty-ninth aspect, wherein the vitrification mixture further comprises an enzyme.

A thirty-second aspect of the present disclosure, either alone or in combination with any other aspect herein, concerns the process of the twenty-ninth aspect, wherein the polynucleotide chain comprises an expression vector.

A thirty-third aspect of the present disclosure, either alone or in combination with any other aspect herein, concerns the process of the thirty-second aspect, wherein the second reagent material comprises an enzyme.

A thirty-fourth aspect of the present disclosure, either alone or in combination with any other aspect herein, concerns the process of any one of the first through fifteenth aspects, wherein biological sample is bound to the substrate.

A thirty-fifth aspect of the present disclosure, either alone or in combination with any other aspect herein, concerns the process of the thirty-fourth aspect, wherein the vitrification mixture further comprises at least one biological sample that is not bound to the substrate.

A thirty-sixth aspect of the present disclosure, either alone or in combination with any other aspect herein, concerns the process of any one of the first through fifteenth aspects, wherein the vitrification mixture further comprises a buffer.

A thirty-seventh aspect of the present disclosure, either alone or in combination with any other aspect herein, concerns the process of any one of the first through fifteenth aspects, further comprising overlying on the substrate a second vitrification mixture after step d) and repeating steps b), c), and d) wherein the second vitrification mixture comprises a second reagent mixture and the vitrification medium on the capillary substrate, wherein the second reagent mixture comprises a second reagent material.

A thirty-eighth aspect of the present disclosure, either alone or in combination with any other aspect herein, concerns a vitrified mixture of two or more materials made by the process of any one of the first through fifteenth or thirty-seventh aspects.

A thirty-ninth aspect of the present disclosure, either alone or in combination with any other aspect herein, concerns the mixture of the thirty-eighth aspect, comprising a biological sample and a second reagent material.

A fortieth aspect of the present disclosure, either alone or in combination with any other aspect herein, concerns the mixture of the thirty-ninth aspect, further comprising a second biological sample.

A forty-first aspect of the present disclosure, either alone or in combination with any other aspect herein, concerns the mixture of the thirty-eighth aspect, wherein the biological sample comprises an enzyme or active fragment thereof.

A forty-second aspect of the present disclosure, either alone or in combination with any other aspect herein, concerns the mixture of the forty-first aspect, wherein the second reagent material comprises a chemical compound that undergoes an enzyme-catalyzed reaction with the enzyme or active fragment thereof.

A forty-third aspect of the present disclosure, either alone or in combination with any other aspect herein, concerns the mixture of the thirty-ninth aspect, wherein the biological sample comprises a first antibody or active fragment thereof.

A forty-fourth aspect of the present disclosure, either alone or in combination with any other aspect herein, concerns the mixture of the forty-third aspect, wherein the second reagent material comprises a second antibody or active fragment thereof, wherein the second antibody or active fragment thereof binds to a different epitope from the first antibody or active fragment thereof.

A forty-fifth aspect of the present disclosure, either alone or in combination with any other aspect herein, concerns the mixture of the thirty-ninth aspect, wherein the biological material comprises a polynucleotide chain.

A forty-sixth aspect of the present disclosure, either alone or in combination with any other aspect herein, concerns the mixture of the forty-fifth aspect, wherein the second reagent material comprises deoxyribonucleotide triphosphate (dNTPs).

A forty-seventh aspect of the present disclosure, either alone or in combination with any other aspect herein, concerns the mixture of the forty-sixth aspect, wherein the mixture further comprises an enzyme.

A forty-eighth aspect of the present disclosure, either alone or in combination with any other aspect herein, concerns the mixture of the forty-fifth aspect, wherein the biological material further comprises a second polynucleotide chain.

A forty-ninth aspect of the present disclosure, either alone or in combination with any other aspect herein, concerns the mixture of the forty-fifth aspect, wherein the polynucleotide chain comprises an expression vector.

A fiftieth aspect of the present disclosure, either alone or in combination with any other aspect herein, concerns the mixture of the forty-fifth aspect, wherein the second reagent material comprises an enzyme.

A fifty-first aspect of the present disclosure, either alone or in combination with any other aspect herein, concerns the mixture of the thirty-ninth aspect, wherein the biological material or second reagent is bound to the substrate.

A fifty-second aspect of the present disclosure, either alone or in combination with any other aspect herein, concerns the mixture of the fifty-first aspect, wherein the first or second reagent material is covalently bound to the capillary substrate.

A fifty-third aspect of the present disclosure, either alone or in combination with any other aspect herein, concerns the mixture of the thirty-ninth aspect, wherein the mixture further comprises a buffer.

A fifty-fourth aspect of the present disclosure, either alone or in combination with any other aspect herein, concerns the mixture of the thirty-eighth aspect, wherein the biological sample is present in said vitrification mixture at 5 μg or less.

A fifty-fifth aspect of the present disclosure, either alone or in combination with any other aspect herein, concerns the mixture of the fifty-fourth aspect, wherein the biological sample is present in said vitrification mixture at equal to or less than 1 μg.

A fifty-sixth aspect of the present disclosure, either alone or in combination with any other aspect herein, concerns the mixture of the fifty-fourth aspect, wherein the biological sample is present in said vitrification mixture at 0.1 μg to 1 μg.

A fifty-seventh aspect of the present disclosure, either alone or in combination with any other aspect herein, concerns the mixture of the fifty-sixth aspect, wherein the total volume of biological sample is from 0.1 μL to 10 μL.

A fifty-eighth aspect of the present disclosure, either alone or in combination with any other aspect herein, concerns the mixture of the thirty-eighth aspect, wherein the vitrification mixture in a volume of 10 μL or less.

A fifty-ninth aspect of the present disclosure, either alone or in combination with any other aspect herein, concerns an assay comprising reconstituting the reagent material of the thirty-eighth aspect with a volume of a solution; and adding a test material thereto, optionally wherein said solution comprises said test material.

A sixtieth aspect of the present disclosure, either alone or in combination with any other aspect herein, concerns a kit comprising a one or more substrates housing a vitrified material made by the process of any one of the first through fifteenth or thirty-seventh aspects.

A sixty-first aspect of the present disclosure, either alone or in combination with any other aspect herein, concerns the kit of the sixtieth aspect, wherein a first capillary substrate comprises the biological material and a second capillary substrate comprises a second reagent material.

A sixty-second aspect of the present disclosure, either alone or in combination with any other aspect herein, concerns the kit of the sixty-first aspect, wherein the vitrified material comprises an enzyme or active fragment thereof.

A sixty-third aspect of the present disclosure, either alone or in combination with any other aspect herein, concerns the kit of the sixty-second aspect, wherein the second reagent material comprises a chemical compound that undergoes an enzyme-catalyzed reaction with the enzyme or active fragment thereof.

A sixty-fourth aspect of the present disclosure, either alone or in combination with any other aspect herein, concerns the kit of the sixty-first aspect, wherein the vitrified material comprises a first antibody or active fragment thereof.

A sixty-fifth aspect of the present disclosure, either alone or in combination with any other aspect herein, concerns the kit of the sixty-fourth aspect, wherein the second reagent material comprises a second antibody or active fragment thereof, wherein the second antibody or active fragment thereof binds to a different epitope from the first antibody or active fragment thereof.

A sixty-sixth aspect of the present disclosure, either alone or in combination with any other aspect herein, concerns the kit of the sixty-first aspect, wherein the vitrified material comprises one or more polynucleotide chains.

A sixty-seventh aspect of the present disclosure, either alone or in combination with any other aspect herein, concerns the kit of the sixty-sixth aspect, wherein the second reagent material comprises deoxyribonucleotide triphosphate (dNTPs).

A sixty-eighth aspect of the present disclosure, either alone or in combination with any other aspect herein, concerns the kit of the sixty-seventh aspect further comprising a third capillary substrate comprising a vitrified enzyme.

A sixty-ninth aspect of the present disclosure, either alone or in combination with any other aspect herein, concerns the kit of the sixty-sixth aspect, wherein the polynucleotide chain comprises an expression vector.

A seventieth aspect of the present disclosure, either alone or in combination with any other aspect herein, concerns the kit of the sixtieth aspect, wherein the biological material is bound to the capillary substrate.

A seventy-first aspect of the present disclosure, either alone or in combination with any other aspect herein, concerns the kit of the seventieth aspect, wherein the biological material is covalently bound to the capillary substrate.

A seventy-second aspect of the present disclosure, either alone or in combination with any other aspect herein, concerns the kit of the sixtieth aspect, wherein the biological sample is vitrified into said capillary substrate at a total mass of 5 μg or less.

A seventy-third aspect of the present disclosure, either alone or in combination with any other aspect herein, concerns the kit of the seventy-second aspect, wherein the biological sample is present in said capillary substrate at a total mass of equal to or less than 1 μg.

A seventy-fourth aspect of the present disclosure, either alone or in combination with any other aspect herein, concerns the kit of the seventy-second aspect, wherein the biological sample is vitrified into said capillary substrate at a total mass of 0.1 μg to 1 μg.

A seventy-fifth aspect of the present disclosure, either alone or in combination with any other aspect herein, concerns the kit of the sixtieth aspect, wherein the total volume of the vitrification mixture is from 0.1 μL to 10 μL.

A seventy-sixth aspect of the present disclosure, either alone or in combination with any other aspect herein, concerns the kit of the sixtieth aspect, wherein the total volume of the vitrification mixture is of 10 μL or less.

While aspects of the disclosure have been illustrated and described, it is not intended that these aspects illustrate and describe all possible forms of the disclosure. Rather, the words used in the specification are words of description rather than limitation, and it is understood that various modifications and substitutions may be made thereto without departing from the spirit and scope of the disclosure.

### PATENT DOCUMENT REFERENCES

| | | |
|---|---|---|
| 6,808,651 B1 | October 2004 | Katagiri, et al. |
| 7,883,664 B2 | February 2011 | G. Elliott; N. Chakraborty. |
| 8,349,252 B2 | January 2013 | G. Elliott; N. Chakraborty. |
| 10,568,318 B2 | February 2020 | P. S. Mohanty, |
| | | N. Chakraborty. |
| US 2013/0157250 A1 | June 2013 | Gutierrez et al. |
| US 2013/0260452 A1 | October 2013 | Toner et al. |

### Non-Patent References

Chakraborty N, Menze M A, Malsam J, Aksan A, Hand S C, et al. (2011) Cryopreservation of Spin-Dried Mammalian Cells, PLoS ONE 6(9): e24916.
Chakraborty N, Biswas D, Elliott G D (2010) A Simple Mechanistic Way to Increase the Survival of Mammalian Cells During Processing for Dry Storage, Biopreservation and Biobanking, 8 (2), 107-114.

Various modifications of the present disclosure, in addition to those shown and described herein, will be apparent to those skilled in the art of the above description. Such modifications are also intended to fall within the scope of the appended claims.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

1. A process for vitrification of one or more biological materials above cryogenic temperature, the process comprising:
a) overlaying a vitrification mixture comprising a biological sample and a vitrification medium on a substrate comprising a capillary network, said substrate in a desiccation chamber;
b) lowering the atmospheric pressure within the desiccation chamber;
c) providing a heat energy from the surface to the vitrification mixture, wherein the heat energy is sufficient to prevent the vitrification mixture from experiencing a freezing condition; and
d) desiccating the vitrification mixture by capillary action until the vitrification mixture enters a glassy state.

2. The process of claim 1, wherein the capillary network is provided by contours along the surface of the substrate.

3. The process of claim 1, wherein the substrate is a wall of the desiccation chamber or is associated with a wall of the desiccation chamber.

4. The process of claim 1, wherein the capillary network within the desiccation chamber is contacted by an underlying solid support substrate.

5. The process of claim 1, wherein vitrification of the vitrification mixture occurs in less than 30 minutes.

6. The process of claim 5, wherein vitrification of the vitrification mixture occurs in less than 10 minutes.

7. The process of claim 1, wherein the heat energy is provided by heating the vitrification mixture.

8. The process of claim 1, wherein the atmospheric pressure is lowered to a value of from about 0.9 atm to about 0.005 atm.

9. The process of claim 8, wherein the atmospheric pressure is lowered to about 0.004 atm.

10. The process of claim 1, wherein the heat energy provided is sufficient to prevent crystallization within the vitrification mixture during vitrification.

11. The process of claim 1, wherein the provided heat energy is sufficient to keep the biological sample at a temperature of from about 0° C. to about 40° C. during said vitrifying.

12. The process of claim 1, wherein said vitrification medium comprises trehalose, glycerol and betine and/or choline.

13. The process of claim 1, wherein the capillary network is hydrophilic.

14. The process of claim 1, wherein the capillary network comprises contiguous capillary channels.

15. The process of claim 1, wherein the biological sample is selected from the group consisting of a nucleic acid, an amino acid, a polynucleotide chain, a peptide, a protein, and an antibody.

16. The process of any one of claims 1-15, wherein said vitrification mixture is overlayed on said substrate at a volume of 10 μL or less.

17. The process of claim 16, wherein the total volume of biological sample is from 0.1 μL to 10 μL.

18. The process of any one of claims 1-15 wherein said biological sample is present is said vitrification mixture at 1 μg or less.

19. The process of claim 18, wherein the biological sample is of a total mass of 5 μg or less, optionally less than 1 μg.

20. The process of claim 19, wherein the biological sample is of a total mass of 0.1 μg to 1 μg.

21. The process of any one of claims 1-15, wherein the vitrification mixture further comprises a second material.

22. The process of claim 21, wherein the second material is a reagent to the biological sample.

23. The process of any one of claims 1-15, wherein the vitrification mixture further comprises a first reagent material.

24. The process of claim 23, wherein the vitrification mixture further comprises a second reagent material.

25. The process of claim 24, wherein the first reagent material comprises an enzyme or active fragment thereof.

26. The process of claim 25, wherein the second reagent material comprises a chemical compound that undergoes an enzyme-catalyzed reaction with the enzyme or active fragment thereof.

27. The process of claim 24, wherein the first reagent material comprises a first antibody or active fragment thereof.

28. The process of claim 27, wherein the second reagent material comprises an antigen or a second antibody or active fragment thereof, wherein the second antibody or active fragment thereof binds to a different epitope from the first antibody or active fragment thereof.

29. The process of claim 24, wherein the first reagent material comprises a polynucleotide chain.

30. The process of claim 29, wherein the second reagent material comprises deoxyribonucleotide triphosphate (dNTPs).

31. The process of claim 29, wherein the vitrification mixture further comprises an enzyme.

32. The process of claim 29, wherein the polynucleotide chain comprises an expression vector.

33. The process of claim 32, wherein the second reagent material comprises an enzyme.

34. The process of any one of claims 1-15, wherein biological sample is bound to the substrate.

35. The process of claim 34, wherein the vitrification mixture further comprises at least one biological sample that is not bound to the substrate.

36. The process of any one of claims 1-15, wherein the vitrification mixture further comprises a buffer.

37. The process of any one of claims 1-15, further comprising overlying on the substrate a second vitrification mixture after step d) and repeating steps b), c), and d) wherein the second vitrification mixture comprises a second reagent mixture and the vitrification medium on the capillary substrate, wherein the second reagent mixture comprises a second reagent material.

38. A vitrified mixture of two or more materials made by the process of any one of claim 1-15 or 37.

39. The mixture of claim 38, comprising a biological sample and a second reagent material.

40. The mixture of claim 39, further comprising a second biological sample.

41. The mixture of claim 38, wherein the biological sample comprises an enzyme or active fragment thereof.

42. The mixture of claim 41, wherein the second reagent material comprises a chemical compound that undergoes an enzyme-catalyzed reaction with the enzyme or active fragment thereof.

43. The mixture of claim 39, wherein the biological sample comprises a first antibody or active fragment thereof.

44. The mixture of claim 43, wherein the second reagent material comprises a second antibody or active fragment thereof, wherein the second antibody or active fragment thereof binds to a different epitope from the first antibody or active fragment thereof.

45. The mixture of claim 39, wherein the biological material comprises a polynucleotide chain.

46. The mixture of claim 45, wherein the second reagent material comprises deoxyribonucleotide triphosphate (dNTPs).

47. The mixture of claim 46, wherein the mixture further comprises an enzyme.

48. The mixture of claim 45, wherein the biological material further comprises a second polynucleotide chain.

49. The mixture of claim 45, wherein the polynucleotide chain comprises an expression vector.

50. The mixture of claim 45, wherein the second reagent material comprises an enzyme.

51. The mixture of claim 39, wherein the biological material or second reagent is bound to the substrate.

52. The mixture of claim 51, wherein the first or second reagent material is covalently bound to the capillary substrate.

53. The mixture of claim 39, wherein the mixture further comprises a buffer.

54. The mixture of claim 38, wherein the biological sample is present in said vitrification mixture at 5 μg or less.

55. The mixture of claim 54, wherein the biological sample is present in said vitrification mixture at equal to or less than 1 μg.

56. The mixture of claim 54, wherein the biological sample is present in said vitrification mixture at 0.1 μg to 1 μg.

57. The mixture of claim 56, wherein the total volume of biological sample is from 0.1 μL to 10 μL.

58. The mixture of claim 38, wherein the vitrification mixture in a volume of 10 μL or less.

59. An assay comprising reconstituting the reagent material of claim 38 with a volume of a solution; and
adding a test material thereto, optionally wherein said solution comprises said test material.

60. A kit comprising a one or more substrates housing a vitrified material made by the process of any one of claim 1-15 or 37.

61. The kit of claim 60, wherein a first capillary substrate comprises the biological material and a second capillary substrate comprises a second reagent material.

62. The kit of claim 61, wherein the vitrified material comprises an enzyme or active fragment thereof.

63. The kit of claim 62, wherein the second reagent material comprises a chemical compound that undergoes an enzyme-catalyzed reaction with the enzyme or active fragment thereof.

64. The kit of claim 61, wherein the vitrified material comprises a first antibody or active fragment thereof.

65. The kit of claim 64, wherein the second reagent material comprises a second antibody or active fragment thereof, wherein the second antibody or active fragment thereof binds to a different epitope from the first antibody or active fragment thereof.

66. The kit of claim 61, wherein the vitrified material comprises one or more polynucleotide chains.

67. The kit of claim 66, wherein the second reagent material comprises deoxyribonucleotide triphosphate (dNTPs).

68. The kit of claim 67 further comprising a third capillary substrate comprising a vitrified enzyme.

69. The kit of claim 66, wherein the polynucleotide chain comprises an expression vector.

70. The kit of claim 60, wherein the biological material is bound to the capillary substrate.

71. The kit of claim 70, wherein the biological material is covalently bound to the capillary substrate.

72. The kit of claim 60, wherein the biological sample is vitrified into said capillary substrate at a total mass of 5 μg or less.

73. The kit of claim 72, wherein the biological sample is present in said capillary substrate at a total mass of equal to or less than 1 μg.

74. The kit of claim 72, wherein the biological sample is vitrified into said capillary substrate at a total mass of 0.1 μg to 1 μg.

75. The kit of claim 60, wherein the total volume of the vitrification mixture is from 0.1 μL to 10 μL.

76. The kit of claim 60, wherein the total volume of the vitrification mixture is of 10 μL or less.
